SEQUENCE LISTING (5 sequences)

SEQ ID NO: 1 - DNA, 693 bp, Mus musculus
tggcggagcccagggatgaagcagctgtgctgttcttgtctgttgtggcttggactcctactgactcctttctccagggaagaggaagaggaatccagacccaggaagctgtgcggcaggcacctgctgatagaagttataaaactctgtggccaaagtgactggagccggttcgagatggaggagcaaagtcctatgacacagttctttccccactactcacgcaagggcaaagccttcaaccctcacccttcttcctccgcctggagaagattcacaaacccagtccctgcaggcgtctctcagaagaaaggaacacacacttgggagcctcagtcactgcccgactatcagtttgaaaagacggagttgcttcctaaggcaagagtgttttcataccacagtggcaagccctatgttaagagcgtacaacttcagaagaaaagcacgaacaaaatgaataccttcagaagtttattttgggggaatcattcccaaaggaaacgcagaggctttgcagataagtgctgtgtgataggatgcaccaaagaagagatggccgtcgcgtgcctcccctttgttgatttttaaaccttaacgattaatcaaacatcactggtgatagagatgtacaaactgtcgtaggaactatgctcgcttaataaaagcttactaaatctaaaaaaaaaaaaa

SEQ ID NO: 2 - protein, 191 aa, Mus musculus
MKQLCCSCLLWLGLLLTPFSREEEEESRPRKLCGRHLLIEVIKLCGQSDWSRFEMEEQSPMTQFFPHYSRKGKAFNPHPSSSAWRRFTNPVPAGVSQKKGTHTWEPQSLPDYQFEKTELLPKARVFSYHSGKPYVKSVQLQKKSTNKMNTFRSLFWGNHSQRKRRGFADKCCVIGCTKEEMAVACLPFVDF

SEQ ID NO: 3 - DNA, 703 bp, Homo sapiens
gcctggggtcacagggatgccgcggctcctccgcttgtccctgctgtggcttggactcctgctggttcggttttctcgtgaactgagcgacatcagcagtgccaggaagctgtgcggcaggtacttggtgaaagaaatagaaaaactctgcggccatgccaactggagccagttccgtttcgaggaggaaacccctttctcacggttgattgcacaggcctcggagaaggtcgaagcctacagcccataccagttcgaaagcccgcaaaccgcttccccggcccggggaagaggcacaaacccagtgtctacttcttgggaagaagcagtaaacagttgggaaatgcagtcactacctgagtataaggataaaaagggatattcaccccttggtaagacaagagaattttcttcatcacataatatcaatgtatatattcatgagaatgcattttttcagaagaaacgtagaaacaaaattaaaaccttaagcaatttgttttgggggcatcatccccaaagaaaacgcagaggatattcagaaaagtgttgtcttacaggatgtacaaaagaagaacttagcattgcatgtcttccatatattgattttaaaaggctaaaggaaaaaagatcatcacttgtaactaagatatactaaccatcttagaattttttctaacctaataaaagcttaatacattta

SEQ ID NO: 4 - protein, 213 aa, Homo sapiens
MPRLLRLTLLWLGLLLVRFSRELSDISSARKLCGRYLVKEIEKLCGHANWSQFRFEEETPFSRLIAQASEKVEAYSPYQFESPQTASPARGRGTNPVSTSWEEAVNSWEMQSLPEYKDKKGYSPLGKTREFSSSHNINVYIHENAFFQKKRRNKIKTLSNLFWGHHPQRKRRGYSEKCCLTGCTKEELSIACLPYIDFKRLKEKRSSLVTKIY

SEQ ID NO: 5 - DNA, 703 bp, Homo sapiens
gcctggggtcacagggatgccgcggctcctccgcttgaccctgctgtggcttggactcctgctggttcggttttctcgtgaactgagcgacatcagcagtgccaggaagctgtgcggcaggtacttggtgaaagaaatagaaaaactctgcggccatgccaactggagccagttccgtttcgaggaggaaacccctttctcacggttgattgcacaggcctcggagaaggtcgaagcctacagcccataccagttcgaaagcccgcaaaccgcttccccggcccggggaagaggcacaaacccagtgtctacttcttgggaagaagcagtaaacagttgggaaatgcagtcactacctgagtataaggataaaaagggatattcaccccttggtaagacaagagaattttcttcatcacataatatcaatgtatatattcatgagaatgcattttttcagaagaaacgtagaaacaaaattaaaaccttaagcaatttgttttgggggcatcatccccaaagaaaacgcagaggatattcagaaaagtgttgtcttacaggatgtacaaaagaagaacttagcattgcatgtcttccatatattgattttaaaaggctaaaggaaaaaagatcatcacttgtaactaagatatactaaccatcttagaattttttctaacctaataaaagcttaatacattta

(19) United States
(12) Patent Application Publication
(10) Pub. No.: US 2003/0229035 A1
(43) Pub. Date: Dec. 11, 2003

(54) Method of restoring ciliated cell motility

(76) Inventors: Ram K. Menon, Pittsburgh, PA (US); Mark A. Sperling, Pittsburgh, PA (US); Chunxia Lu, Pittsburgh, PA (US); Selma Witchel, Pittsburgh, PA (US); John Kasik, Santa Maria, CA (US)

Correspondence Address: CRAIG G. COCHENOUR, BUCHANAN INGERSOLL, P.C., ONE OXFORD CENTRE, 20th FLOOR, 301 GRANT STREET, PITTSBURGH, PA, 15219, US

(21) Appl. No.: 09/967,399

(22) Filed: Sep. 28, 2001

(51) Int. Cl. A61K 48/00

(52) U.S. Cl. 514/44; 800/21

(57) ABSTRACT

The present invention relates to a novel gene from the insulin family, Insl6, which expresses a protein restoring motility in ciliated cells. The proteins of the insulin family play essential roles in pleiotropic physiological processes affecting metabolism, growth, and reproduction. A new member of the insulin family named Insl6 is disclosed playing an essential role in ciliated cell activity. Insl6 plays an essential role in spermatocyte function. Thus, the Insl6 gene and its protein product are useful in the treatment of infertility caused by the loss of spermatocyte motility. A method of modulating male fertility is disclosed.

### BACKGROUND OF THE INVENTION

1. Field of the Invention

The present invention relates to a novel method of administering or regulating a gene from the insulin family, INSL6, or its protein product restoring motility in ciliated cells. The proteins of the insulin family play essential roles in pleiotropic physiological processes affecting metabolism, growth, and reproduction. The application of the techniques of molecular and computational biology has resulted in the identification of newer members of this family of proteins. A new member of the insulin family named Insl6 is disclosed playing an essential role in ciliated cell activity. Specifically, Insl6 plays an essential role in spermatocyte function. Thus, the INSL6 gene and its protein product are useful in the treatment of infertility caused by the loss of spermatocyte motility.

2. Brief Descirption of the Background Art

The insulin/IGF/relaxin family encompasses a group of functionally diverse proteins. Despite the magnitude of functional divergence present within the family, all members of the insulin family exhibit a high degree of structural conservation. It is an ancient family of proteins. Insulin or insulin-like proteins have been described in unicellular eukaryotes as well as in such primitive species as insects, tunicates, annelids, and molluscans

The primary peptide sequence of each member of the family is characterized by three domains comprising an amino terminal B peptide (or chain) joined to a carboxyl A peptide by an intervening C peptide (B-C-A). The B and A chain peptides are relatively invariant between the different hormones of the family and between species for a specific hormone, and exhibit a pattern of distinct and highly conserved cysteine motifs. These cysteine motifs characterize the family. Specifically, the cysteine motif present in the A peptide has been termed the insulin signature. Insulin family hormones are synthesized as prehormones and for the majority of members of the family, the primary peptide undergoes post translational modification to generate a cysteine-linked heterodimer of the B and A peptides that functions as the active hormone.

In mammals, the insulin family has till recently been comprised of five members: insulin, the insulin-like growth factors (Igf IA, Igf IB and Igf II), and relaxin. Recent application of the tools of molecular and computational biology has resulted in rapid identification of four new members of the insulin family termed INSL3, INSL4, INSL5, and INSL6. Henceforth, the INSL6 abbreviation is used to designate the human Insulin 6 gene, and the abbreviation INSL6 is used to designate the human Insulin 6 protein. The mouse or rat Insulin 6 gene are designated by Insl6 for the gene, and Insl6 for the protein.

The mammalian insulin gene (INS) is principally expressed in the pancreas and its biological functions are metabolism and growth. In the mouse, the Ins1 pseudogene and the Ins2 gene are also expressed in the pancreas and have principal biological functions in metabolism and growth. Two insulin-like growth factors I and II, IGF1 and IGF2 respectively, are principally expressed in the liver. The major biological function of IGF1 is growth, and the putative function of IGF2 is fetal growth. The relaxin H1 and relaxin H2 genes, RLN1 and RLN2 respectively, are principally expressed in the corpus luteum and play a role in parturition.

The relaxin-like factor INSL3 is principally expressed in the testes and plays a role in testicular descent in male fetuses. Another member of the insulin family recently identified, the early-placenta insulin-like peptide INSL4, also known as placentin, is expressed in the placenta. Placentin was identified by differential expression techniques. Another insulin-like protein, INSL5 is also known as relaxin/insulin-like factor 2 and is expressed in the GI tract and the kidney. The major biological functions for INSL4 and INSL5 have not yet been elucidated.

The product of the INSL3 gene is also termed the relaxin-like factor (RLF) or Leydig insulin-like peptide precursor (Ley-I-L). The INSL3 gene was initially identified in boar testes but was subsequently identified in both the human and the mouse. The major site of expression of INSL3 is in Leydig cells within the testes with expression also being detected in theca cells of the corpus luteum, the trophoblast and breast. In the testes, INSL3 is a specific marker for the mature Leydig cell phenotype. In mice, the INSL3 gene plays a major role in the development of the gubernaculum and subsequent testicular development. Male mice homozygous for targeted disruption of the INSL3 gene are cryptorchid wheareas the female homozygotic deletion mutants experience disruption of the estrus cycle with resultant problems with fertility. Furthermore, mutations in the INSL3 gene have been demonstrated in a subset of patients with cryptorchidism. Within the B peptide, INSL3 retains the R-X-X-X-R motif implicated in relaxin ligand-receptor interactions. INSL3 competes with relaxin for binding to the relaxin receptor, although with a lower affinity. INSL3 also binds to a second distinct receptor that is specific for INSL3. In the symphysis pubis relaxation assay, INSL3 exhibits relaxin-like softening properties, but only in synergy with relaxin and not independently. INSL3 circulates systematically suggesting a hormonal role in addition to paracrine and/or autocrine functions.

The product of the INSL4 gene is also known as the early placenta insulin-like peptide, EPIL or placentin and was identified as a differentially expressed gene within the human placenta. Mouse or rat orthologs of INSL4 have not been identified. The predominant site of expression of the INSL4 gene is in the placenta during early pregnancy, but it is also expressed to a lesser degree in interbone ligaments, perichondrium and the uterus. INSL4 is detectable in maternal serum during pregnancy and presumably functions as a hormone, as well as exhibiting paracrine and/or autocrine actions. There is a paucity of information regarding possible receptors for the product of INSL 4, although conditioned media from cells transfected with the INSL4 cDNA shows functional activity through a putative receptor that is distinct from the insulin receptor.

The INSL5 gene was identified through the application of the techniques of computational biology, also known as in silico cloning. Mouse and rat orthologs of INSL5 have been identified. The human INSL5 gene is located on chromosome 1 and the orthologous mouse INSL5 is located on mouse chromosome 4. Expression of human INSL5 has been demonstrated in the uterus and the digestive tract with highest levels of expression in the rectum. Expression of mouse INSL5 is described in thymus, kidney, heart, brain and testis.

Many members of the insulin family undergo post-translational modification to produce the biologically active hormone. The major endoproteolytic processing enzymes of the secretory pathway belong to the subtilisin-like pro-protein convertase (PC or SPC) family. This family of proteins include furin (also termed SPCI/PACE), PC2/SPC2, PC1/PC3/SPC3, PACE4/SPC4, PC4/SPC5, PC6/SPC6, and PC7/SPC7/PC8/LPC. In the prototypic example of insulin the signal peptide is proteolytically cleaved, the C-peptide is excised by prohormone convertases (PC 1/PC3 and PC2), and the B- and A-domains are linked by inter- and intra-disulfide bonds to produce the biologically active form of insulin. PC4 is a member of this proprotein convertase family of serine proteases. In rodents, PC4 transcripts are expressed in spermatocytes and round spermatids exclusively. The phenotype of PC4 null mice is characterized by male infertility due to a functional abnormality in sperm motility and early embryonic death. PC4 and Insl6 are co-expressed in the same cell types in the testes, and have a congruent ontogenic profile of expression in germ cells of the testes. There is thus a causative link between PC4 deficiency and infertility in male mice, and Insl6 which can restore fertility in PC4 null mice.

The INSL6 gene plays a role in modulating the functioning of the Sertoli cell. The follicle stimulating hormone (FSH) is one of the major regulators of Sertoli cell number and function. The response of the Sertoli cell to FSH varies with the development status of the animal. In general the actions of FSH on the Sertoli cell can be grouped into those affecting the prenatal and newborn animal, those affecting the immature testis (in the rat this corresponds to 10-30 day old rats), and those affecting the adult mature testis (in the rat this corresponds to greater than 40 days postnatal age).

The insulin family of genes have wide ranging effects in essential functions ranging from metabolism and growth to embryonic development of the testis in the male fetus. There is a significant need for an activation or development promoter factor exerting activation of ciliated cells, specifically spermatocytes, useful in the treatment of male infertility caused by impaired spermatocyte motility. The gene and method of the present invention and its expression product INSL6 provide a means to activate ciliated cells, specifically spermatocytes, and are useful in the treatment of male infertility in humans. Conversely, deactivation or regulation of the INSL6 gene is useful in modulating male fertility both in vivo and in vitro.

### SUMMARY OF THE INVENTION

A method of use of a new member of the insulin family of proteins, termed Insl6, and its gene INSL6, are disclosed. The INSL6 protein is highly expressed in germ cells of the testis. Evolutionary conservation of the structure of this gene and protein suggests that it plays an important regulatory biological role on Sertoli cells, and ciliated cells such as sperm. INSL6 exerts its biological activity by modifying the function of the Sertoli cell. However, INSL6 may have other targets of action. Thus INSL6 has both an autocrine role for with effects on germ cells, and an endocrine role with effects on pituitary release of FSH. The biological role of INSL6 is to regulate Sertoli cell function. The INSL6 gene and its expressed protein INSL6 are useful to ameliorate pathological states such as infertility by altering the expression/function of this protein. The present invention is directed to the methods of use of the INSL6 protein and INSL6 gene to modulate pathological states resulting in male infertility.

The methods of the present invention are directed to modulating the INSL6 gene which results in the modulation of sperm motility and thus modulation of fertility in male subjects with male infertility caused by impaired sperm motility or capacitance. Conversely, inhibition of the Insl6 functions on Sertoli cells is useful in modulating fertility in normal subjects to reduce sperm motility and capacitance resulting in induced infertility in male subjects. The INSL6 gene can be modulated by controlling its expression in vivo by controlling repressor or promoters of expression, or by inhibiting expression by anti-sense nucleic acids. Alternatively, INSL6 can be controlled in vitro by treating sperm from a subject having male infertility caused by impaired sperm motility or capacitance.

### BRIEF DESCRIPTION OF THE DRAWINGS

FIG. 1 shows the sequence of cDNA for INSL6 (GenBank accession AF 143807)

FIG. 2 shows a comparison of B and A peptides and contiguous amino acid sequence for mouse (m), rat (r), and human (h) Insl6.

FIG. 3 shows the INSL6 gene mapping to the Central Part of Mouse Chromosome 19.

FIG. 4 shows CHO cells stably expressing INSL6 and detection of INSL6 in conditioned medium.

FIGS. 5A-E show post-translational modification of INSL6.

FIG. 6A shows Northern blot analysis of expression of INSL6.

FIG. 6B shows Western blot analysis of anti-Insl6 antibody.

FIG. 7 shows INSL6 expression and tissue distribution with highest expression in the testis.

FIG. 8 shows ontogeny of Insl6 expression in mouse testis.

FIG. 9A shows cyclic AMP-mediated increase in INSL6 expression in rat primary germ cells.

FIG. 9B shows the construct map of the adenoviral vector expressing the epitope-tagged murine Insl6 protein.

FIG. 10 shows adenovirally mediated expression of INSL6.

FIG. 11A shows Insl6 stimulation of Tyrosine phosphorylation of Proteins in Sertoli cells.

FIG. 11B shows the presence of two species of Insl6, 31 kDa and 40 kDa.

FIG. 11C shows that Insl6 is ubiquinated.

FIG. 12 shows effects of Insl6 on proliferation of MSC-1 cells.

FIG. 13 shows a Single Strand Conformational Polymorphism (SCCP) Analysis of INSL6 in patients with decreased sperm motility by SSCP autoradiograph gel from exon 1 of the INSL6 gene in five patients with infertility characterized by decreased sperm motility. The unique conformer in patient #2 is boxed in white.

FIG. 14 shows Restriction Analysis to Confirm the 22T to A Nucleotide Change in Codon 8 of the INSL6 gene.

FIG. 15 shows the 22T to A mutation in INSL6 gene that results in reduced efficiency of translocation in the endoplasmic reticulum.

### DETAILED DESCRIPTION OF A PREFERRED EMBODIMENT

As used herein, the term “modulating” as it relates to the INSL6 gene means up-regulating or down-regulating the INSL6 gene or its protein product.

As used herein, the term “modulating” as it relates to sperm motility, fertility, and/or capacitance, means decreasing or increasing the same.

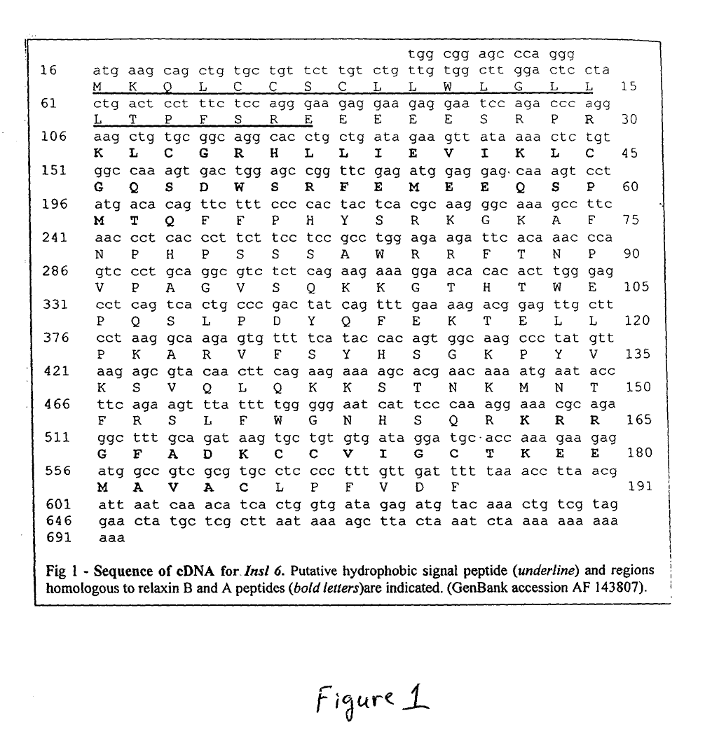

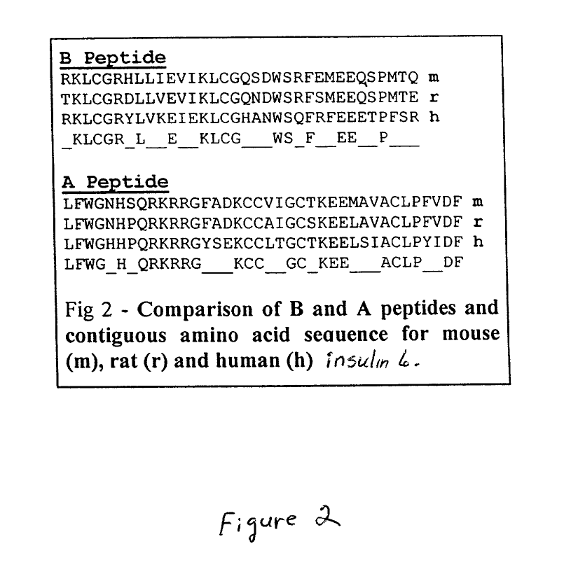

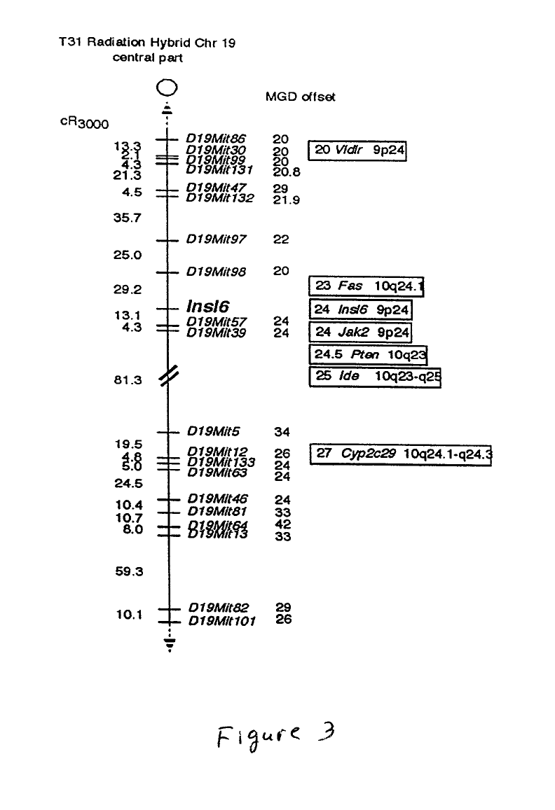

The coding sequence or cDNA for INSL6 is approximately 700 base pairs (bp). The cDNA has a single open reading frame that encodes a protein approximately 22,000 daltons containing 191 amino acids (FIG. 1). Analysis of the amino end of the protein suggests the presence of a hydrophobic signal peptide with a cleavage site between amino acids 23 and 24. The protein contains a B peptide, a connecting (C) peptide and an A peptide (FIG. 2). Within all members of the insulin family, the cysteine motif with its characteristic spacing is highly conserved within the B an A domains. Thus the B-domain motif is defined as LCG X₁₀C where XN represents the number of residues comprising any amino acids other than cysteine and the A-domain is defined as CCX₃CX₈C. The B and A peptides of INSL6 fulfill these consensus criteria. Flanking the amino end of the B peptide is the peptide R-K-L-C-G-R (amino acids 30-35) that approximates the R-X-X-X-R cassette found in the B peptides of relaxin and other relaxin-related proteins. This cassette is implicated as a recognition site for the relaxin receptor.

There is a high degree of conservation of sequence found within both the A and B peptides of mouse, human and rat Insl6 (FIG. 2). As is frequently observed among insulin family members, there is not a high degree of conservation of sequence between rodent and human connecting (C) peptides, but as expected for orthologous genes, there is a high degree of conservation of sequence present in the rodent connecting peptides. A comparison of the mouse protein against GenBank (blastp) suggests that the mouse Insl6 protein is similar to relaxin.

The chromosomal location of INSL6 was mapped by radiation hybrid mapping. Primers were designed that amplified both mouse cDNA as well as mouse genomic DNA (entire amplicon resides within an exon). The INSL6 amplicon was 129 bp in length (F:AGAGATGGCCGTCGCGTG, R:AGATTTAGAGTAAGCTTTTATTAAGCGAGC). The INSL6 primer set gave differential amplification of mouse genomic vs. chinese hamster ovary (CHO) genomic DNA. The Jackson Laboratory's T31 mouse radiation hybrid panel (100 hybrid cell lines as well as mouse and CHO controls) was screened with the Insl6 primer set in duplicate and linkage analysis performed using the Jackson Laboratory's database. The location of Insl6 gene was mapped to mouse chromosome 19 (between D19Mit57 and D19Mit98) (FIG. 3). Insl6 is approximately 13.1cR centromeric of D19Mit57. FIG. 3 illustrates the mouse chromosome 19 ideogram with the Insl6 gene and its immediate neighbors indicated as well as mapped human orthologs. It is noteworthy that human INSL6 maps to 9p24, a region syntenic to mouse chromosome 19 (MGD 24) where the mouse Insl6 gene resides.

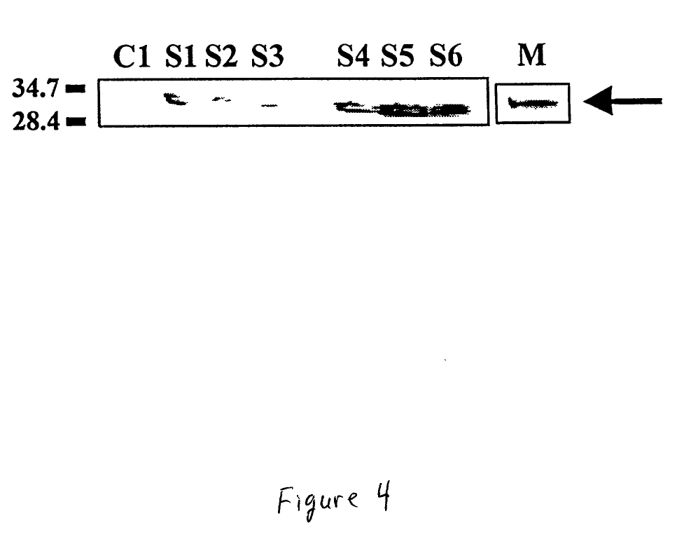

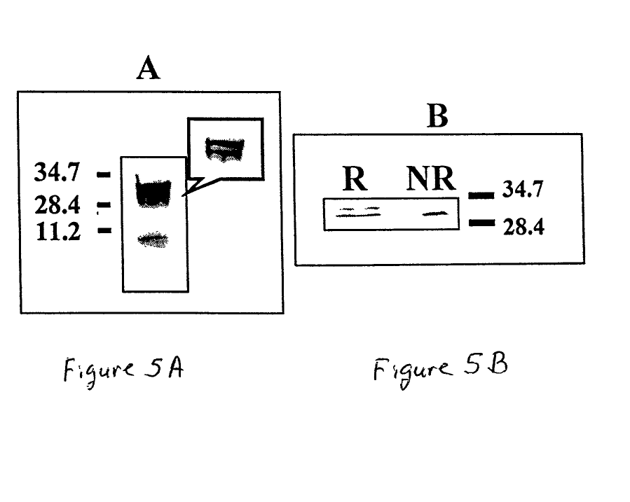

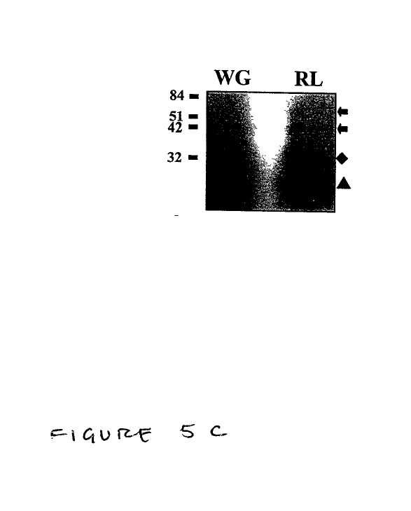

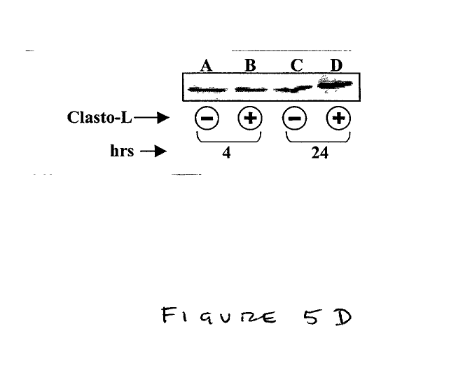

To enable investigations into the structure and function of INSL6, a stable cell line expressing an epitope-tagged Insl6 fusion protein was designed. For this purpose, the flip-In (Invitrogen, Inc.) strategy was utilized to transfect competent CHO cells with a plasmid coding for Insl6 fusion protein tagged at its c-terminal with a His-myc epitope. Monoclonal cell lines that expressed Insl6 were selected by dilutional cloning. The expression of the epitope-tagged Insl6 protein was verified by Western blot analysis. Furthermore, analysis of the supernatant revealed that the fusion protein is secreted into the culture medium (FIG. 4). Another expression system was used to obtain adequate quantities of Insl6. The expression system used was (Bac-to-Bac; Life Technologies). This expression system was constructed by taking a recombinant bacmid containing an expression cassette directing expression of His-myc epitope-tagged murine Insl6 using the polyhedrin promoter from Autographa californica nuclear polyhedrosis virus. Western blot analysis of Spodoptera frugiperda (Sf9) insect cells infected with this recombinant bacmid (pFastBac 1-mInsl6), confirmed the expression of Insl6 in these cells (FIG. 5). It is of interest to note that the size of Insl6 molecule expressed in Sf9 cell is approximately 40 kDa. Possible explanations for the difference in molecular weight from that of Insl6 expressed in CHO cells, approximately 31 kDa, are the different possible splicing observed with intronless Insl6 expression cassette, or alternatively different post-translational modification. To obtain partially purified recombinant Insl6 protein, the His-myc epitope tagged Ins16 protein was purified under native conditions from the Sf9 lysates using NiTA affinity columns (Qiagen). The yield from 1 liter of Sf9 cells was approximately 5-10 μg of Insl6. The recombinant Insl6 was used to test the effects on rat primary Sertoli cells.

Many members of the insulin family undergo post-translational modification to produce the biologically active hormone. Thus, in the prototypic example of insulin the signal peptide is proteolytically cleaved, the C-peptide is excised by prohormone convertases (PC1/PC3 and PC2), and the B- and A-domains are linked by inter- and intra-disulfide bonds to produce the biologically active form of insulin. Prohormone protein convertases constitute a family of serine proteases structurally related to bacterial subtilisins and to yeast kexin. Several eukaryotic members of this family are currently known. Prohormone Convertases (PC's) cleave precursor polypeptides at specific basic residues, most often after selected paired basic residues, to generate bioactive peptide and proteins. Many members of the insulin family of proteins (e.g. Insulin, Igf-1) are substrates for PC's. Examination of Western blots of CHO cells expressing the epitope-tagged Insl6 protein allowed us to make deductions regarding post-translational modifications of Insl6 based on the configuration that the myc-epitope is attached at the C-terminus of Insl6 and thus linked to the putative A-domain. Under non-reducing conditions (FIG. 5B) when cell lysates of CHO cells stably expressing Insl6 were subjected to treatment of the cell lysates with the reducing agent β-mercaptoethanol (FIG. 5A), three bands of approximately 32, 30, and 10 kDa were detected using an anti-myc antibody. These bands most likely represent B-C-A, C-A, and A peptides respectively, suggesting that analogous to insulin, Insl6 also undergoes post-translational processing. It is noteworthy that the testis is the site of expression of proconvertase PC4. Furthermore, the cell types (i.e. pachytene spermatocytes and round spermatids), in which Insl6 is expressed are the same as those in which PC4 is expressed. At present the natural substrate(s) for PC4 are not well defined. The co-localization of expression of Insl6 and PC4 makes Insl6 a likely candidate substrate for PC4.

Based on the magnitude of difference in molecular weights between the two species (31-32 vs. 40 kDa), it was investigated whether Insl6 is ubiquinated. Coupled in vitro transcription/translation using reticulocyte lysates revealed protein bands of 39-41 and 46-48 kDa in addition to the expected band of 31-32 kDa (FIG. 5C). In contrast, the absence of the larger bands in a transcription/translation reaction using wheat germ extracts (FIG. 5C) supports the possibility that the Insl6 protein is ubiquinated. This was further corroborated by demonstration that exposure to CHO cells stably expressing recombinant Insl6 to a proteosome inhibitor (clasto-lactacystin-β-lactone) resulted in prolongation of the half-life of Insl6 indicating that Insl6 is degraded via the proteosome pathway (FIG. 5D). Briefly CHO cells stably expressing Insl6 were either exposed to vehicle (−) or 5 μM Clasto-Lactacystin-β-lactone (Clasto-L) (+) and cells harvested after 4 h (lanes A & B) or 24 h (lanes C & D). The cell lysates were Western blotted with anti-Insl6 antibody. To address te possibility that over-expression of Insl6 in the CHO cells may have facilitated aberrant ubiquination, the effect of inhibition of the proteasome pathway in a coupled in vitro transcriptiontranslation was investigated. The data obtained support the in vivo data showing that inhibition of the proteasome pathway results in decelerated rate of degradation of the Insl6 (FIG. 5E). Briefly, coupled in vitro transcription/translation of Insl6 using reticulocyte lysates performed in the presence of [³⁵S] L-cysteine and either vehicle(−) or 5 μM Clasto-Lactacystin-β-lactone (Clasto-L) (+) showed that inhibition of the proteasome pathway in a coupled transcription/translation system resulted in decelerated rate of degradation of Insl6 (FIG. 5E).

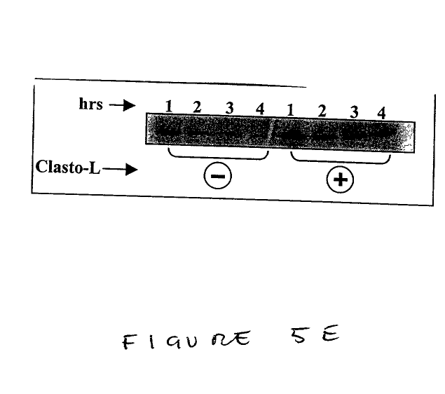

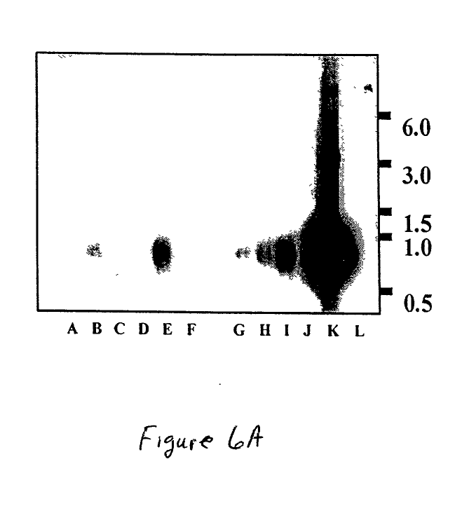

Northern blot analysis was utilized to ascertain the profile of Insl6 expression. The Northern blot analysis was conducted by using oligo dT selected murine RNA (FIG. 6A). A specific signal (0.6-0.8 kB), consistent with the size of the cDNA, was detected. Expression was minimized in the testes, with lower levels of mRNA detected in kidney, small bowel, heart, brain, in situ hybridization revealed that Insl6 expression was restricted to the olfactory & vomeronasal epithelium.

To further facilitate investigations into the biological role of Insl6, antibodies against Insl6 were generated. Towards this end, two peptides belonging to the C chain were designed and synthesized. Following co-immunization of rabbits with these two peptides, the serum was affinity purified and tested using Western blotting. Briefly, anti-Insl6 antibody was used in Western blot, under reducing conditions SDS-PAGE, of cell lysates of CHO cells stably expressing Insl6. FIG. 6B shows that the specificity of the detected signal was ascertained by determination of the molecular weight and by competition with molar excess of immunizing peptide at ox (lane A, FIG. 6B), 100× (lane B, FIG. 6B), or 250× (lane C, FIG. 6B) concentrations. These results indicate that the anti-Insl6 antiserum is sufficiently specific and of an adequate titer to perform Western blotting and quantitation of Insl6 expression. This analysis indicate that antiserum of a reasonable titer had been obtained (FIG. 6B).

In another embodiment of this invention, an isolated an purified antibody or fragment thereof which specifically binds to a polypeptide of SEQ ID Nos. 2 or 4 is provided. The antibody may be a polyclonal or monoclonal antibody. Yet another embodiment of this invention provides for a hybridoma cell line which produces the monoclonal antibody.

In another embodiment of this invention, a kit is provided comprising (a) the antibody or fragment described herein and (b) a detection reagent to detect the presence of a specifically bound antibody. This invention includes a composition comprising the antibody or fragment thereof described herein and a pharmaceutically acceptable carrier, excipient or diluent. The kit's detection reagent is a radioisotope, affinity label, enzymatic label, fluorescent label, or paramagnetic label. The present invention includes a method of detecting the presence of an INSL6 polypeptide having the amino acid sequence of SEQ ID No. 4 or a fragment thereof in a cell, tissue, or fluid sample, comprising: (a) contacting the cell, tissue, or fluid sample with an antibody or fragment as described herein under conditions that allow the formation of an antibody/polypeptide complex, and (b) detecting the presence of an antibody polypeptide complex, wherein the presence of the antibody/polypeptide complex indicates the presence of an INSL6 polypeptide.

The present invention includes a method for modulating male fertility comprising modulating cyclic-AMP levels for modulating INSL6 activity in sperm cells. This method includes wherein the cyclic AMP level is modulated in vivo or in vitro. Further, the present invention includes a method for modulating male fertility comprising modulating a proteasome pathway for modulating INSL6 activity. This method includes wherein the proteasome pathway is modulated in vivo or in vitro.

in another embodiment of this invention, a method of diagnosing a mutation or deletion in a the INSL6 sequence of a subject is provided comprising determining allelic homozygocity or heterozygocity of said subjects INSL6 by hybridizing in a Southern blot with marker probe selected from the group consisting of D9S251, D9S248, D9S256, DS1686, D9S1873, D9S1792, and D9S1858.

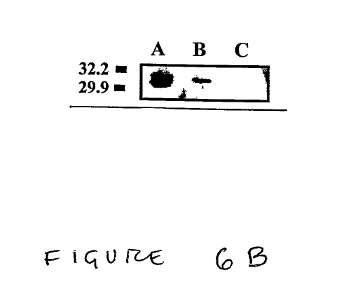

To facilitate the quantitative measurement of Insl6 expression a real time RT-PCR (TaqMan) (PE Biosystems, Foster City, Calif.) assay was developed and standardized to quantify Insl6 MRNA levels in various murine tissues (FIG. 7). These studies confirmed that Insl6 was maximally expressed in the testis with abundance of Insl6 MRNA as shown in FIG. 7 in the small bowel (B) (8%), kidney (C) (2%), uterus (D) (2%), ovary (E)(1%), spleen (F)(1%), breast (G)(0.5%), and thymus (H)(0.5%) being significantly less than in the testis (A) (100%). TaqMan analysis also revealed that Insl6 mRNA is abundant in primary cultures of germ cells, but is not detected in Sertoli cells or Sertoli cell line (MSC-1). These data support known observations that the primary site of expression of Insl6 in the testis is in pachytene spermatocytes and round spermatids and not in Leydig cells.

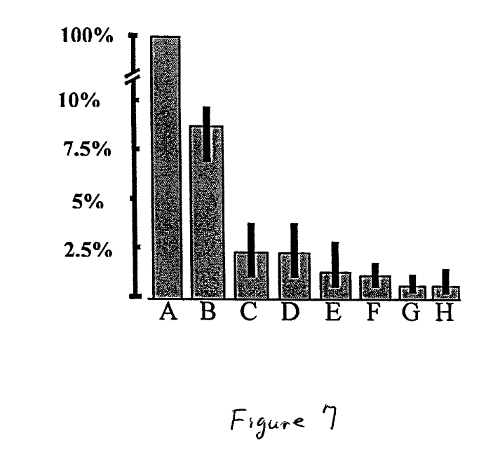

Examination of the ontogenic profile of Insl6 expression in mouse testis revealed that Insl6 MRNA was detectable at low levels at embryonic day 14.5 and 17.5, and the level of expression remained low (day zero and seven days postnatal) until postnatal day twenty when there was a twenty (20) fold increase in Insl6 MRNA abundance with maximal levels attained by forty (40) days postnatal and maintained up to ninety (90) days of age as set forth in FIG. 8. Pachytene spermatocytes, a major site of expression of Insl6 first appear in the mouse testis around 20 days of age. FIG. 8 shows the expression of Insl6 mRNA as measured by taqMan assay in testis of mice at indicated ages. The values in FIG. 8 are represented as “fold increase” relative to the level of Insl6 mRNA at embryonic (e) 14.5 days. In FIG. 8, postnatal is designated “p”.

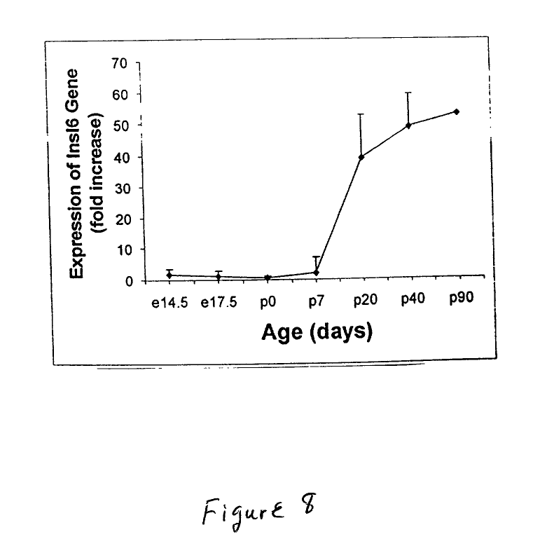

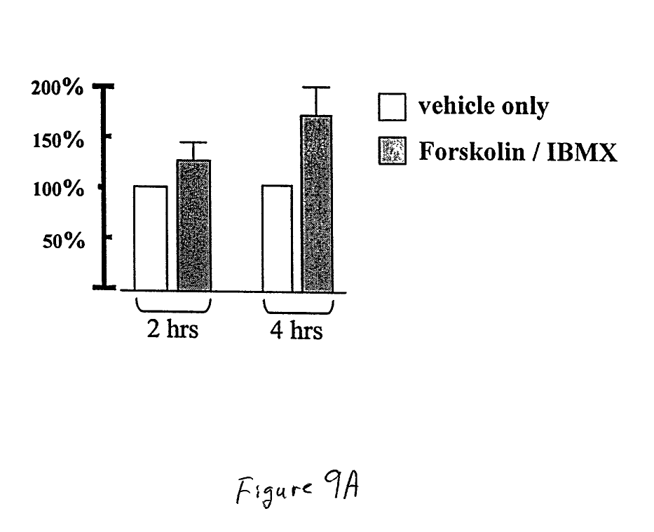

Cyclic-AMP (cAMP) is a major intracellular regulator of gene expression in the testis. To begin to understand the factors that regulate expression of Insl6, the effects of cAMP on Insl6 were investigated. For these experiments, a real time quantitative assay (TaqMan) was standardized for measurement of rat Insl6. Rat primary germ cells in culture were exposed to forskolin/IBMX mixture for varying time periods and changes in Insl6 gene expression monitored by TaqMan assay based estimation of mRNA levels of rat Insl6 and of the housekeeping gene 18S. These results indicated that exposure of germ cells to forskolin/IBMX significantly increases the expression of Insl6 mRNA (FIG. 9A). This effect was observable by 2 hours and increased in magnitude after 4 hours of exposure to forskolin/IBMX mixture.

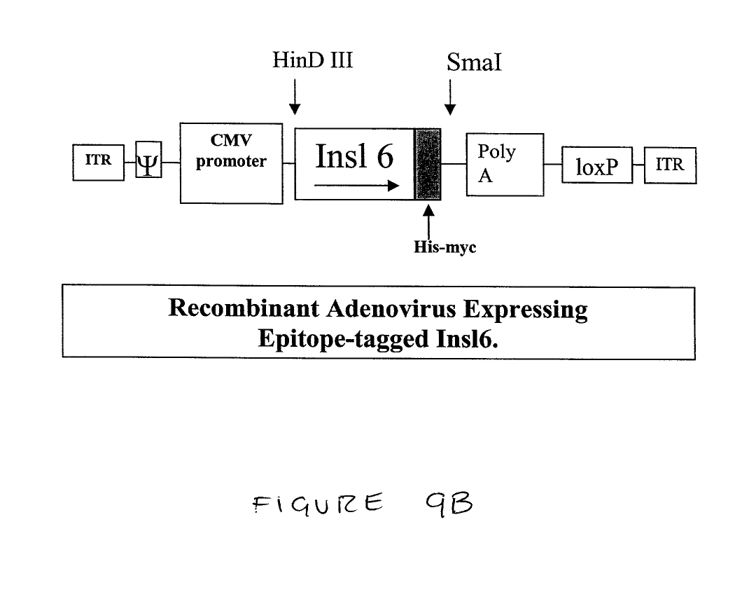

To facilitate the study of the function of Insl6 protein both in vitro and in vivo, a recombination-deficient adenoviral vector expressing the epitope-tagged murine Insl6 protein was engineered (FIG. 9B). For this purpose the second generation shuttle vector pADLOX was modified to contain the murine Insl6 cDNA (Insl6Adlox). The production of the replication-deficient adenoviral vectors expressing the Insl6 cDNA was achieved by a protocol based on the Cre-lox principle for efficient and relatively quick identification of recombinant viral particles. The adenovirus was plaque purified and a cesium chloride preparation used for the experiments. Western blot analysis of human embryonal kidney (293) cells infected with the recombinant adenovirus confirmed the expression of myc-tagged Insl6 fusion protein (FIG. 10).

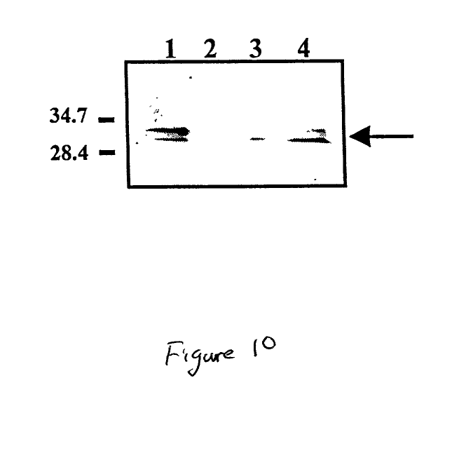

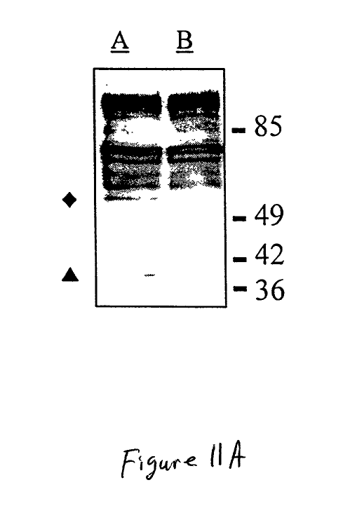

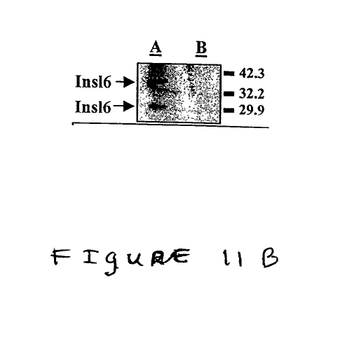

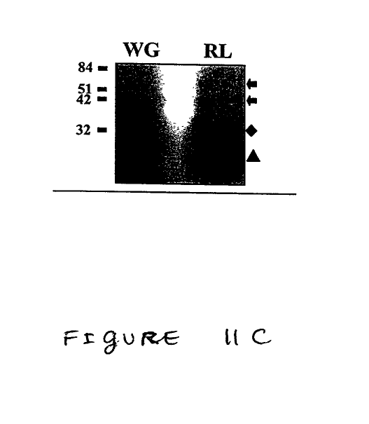

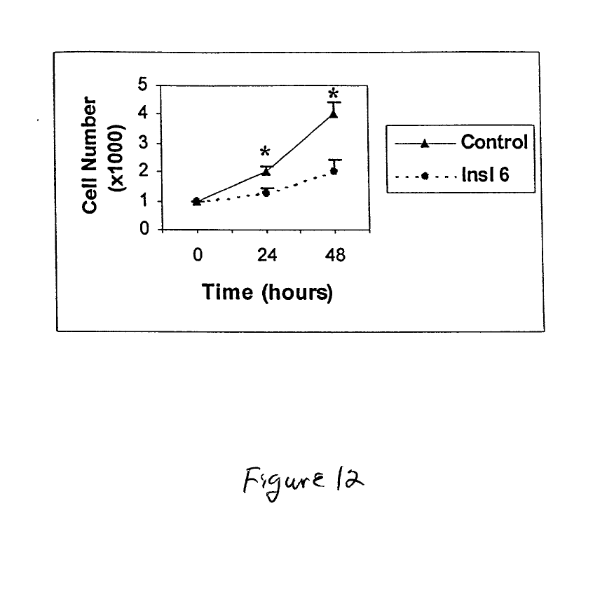

The effect of Insl6 on Sertoli cells was confirmed by exposing serum-deprived MSC-1 (mouse Sertoli cell line) cells to serum-free conditioned medium containing recombinant Insl6 secreted by the CHO cells stably expressing epitope-tagged Ins16. The effect of Insl6 on the phosphorylation status of MSC-1 cellular proteins was initially investigated. These studies revealed that exposure to recombinant Insl6 resulted in specific tyrosine phosphorylation of proteins of molecular weights of approximately 55 and 40 kDa (FIG. 11A). Serum-deprived MSC-1 cells were exposed for ten minutes to serum-free conditioned medium from either CHO cells expressing Insl6 (lane A, FIG. 11A) or from non-transfected CHO cells (lane B, FIG. 11A). Cell lysates were subjected to reducing SDS-PAGE and blotted with antiphphosphotyrosine antibodies. The positions of molecular weight standards are indicated in FIG. 11A. The specific protein bands showing increased phosphorylation in response to Insl6 are indicated by the diamond and star symbols respectively. These results were consistently demonstrable on multiple occasions.

To further investigate the significance of alteration in the tyrosine phosphorylation status of MSC-1 proteins, the effects of Insl6 on proliferation of MSC-1 were tested. For these experiments serum-deprived MSC-1 cells were exposed for varying time periods to serum-free conditioned medium from either CHO cells expressing Insl6 or from non-transfected CHO cells and the number of viable cells determined by a colorimetic assay (celltiter 96 Aqueous One Solution Cell Proliferation Assay; Promega, Madison, Wis., USA). These experiments established that Insl6 significantly inhibits the proliferation of MSC-1 in a time-dependent manner, providing further proof that the Sertoli cell is a target of action of Insl6.

Insl6 modulates the function of the Sertoli cell. Insl6 expression is greatest in the germ cells of the testis. Our data show that recombinant Insl6 alters the tyrosine phosphorylation status of Sertoli cell proteins and decreases the proliferation of Sertoli cells. Insl6 modulates Sertoli cell function in three principal ways: (i) modulation of function of the Sertoli cell by FSH, (ii) synthesis of Sertoli cell proteins, and (iii) canonical signaling pathways in the Sertoli cell.

Insl6 Paracrine Effect—Germ Cell Secretion

The secretion of Insl6 by germ cells exerts paracrine effect on the Sertoli cells. This is demonstrated by the detection of Insl6 in the culture medium of CHO cells stably expressing recombinant Insl6. This was demonstrated in vivo by detecting Insl6 in seminiferous tubule fluid in adult rats. Briefly, following anesthetization of the animal, the testis were exposed via a scrotal incision and portions of the tunic albuginea dissected to reveal the underlying tubules. To maintain the correct physiological temperature of the testis and to prevent dehydration, the tissue is covered with warm mineral oil. The seminiferous tubule was collected from the rete testis and centrifuged to remove spermatozoa. The supernatant was subjected to ELISA using the anti-Insl6 antibody previously described, or alternatively to radioimmunoassay, to detect the presence of Insl6 protein. These assays showed the presence of two species of Insl6 (31/32 and 40 kDa) in the rodent testis (FIG. 1I B). These results suggested post translational modification such as ubiquination (FIG. 1C). Briefly, coupled in vitro transcription/translation using either wheat germ extracts (WG) or reticulocytes lysates (RL) performed in the presence of [35S] L-cysteine. The reaction products were size-fractionated via SDS-PAGE and autoradiographed. The results suggest that Insl6 is ubiquinated. Many proteins secreted into the seminiferous tubule fluid enter the systemic circulation. A canonical example is that of inhibin which is secreted into the seminiferous tubule fluid and is the resorbed into the systemic circulation from the rete testis via the overlying mediastinal venous plexus.

Effects of Insl6 on the Actions of FSH on the Sertoli Cell

Insl6 plays a role in modulating the functioning of the Sertoli cell. FSH is one of the major regulators of Sertoli cell number and function. The response of the Sertoli cell to FSH varies with the developmental status of the animal. In general the actions of FSH on the Sertoli cell can be grouped into those effecting the prenatal and newborn animal, those effecting the immature testis (in the rat this corresponds to 10-30 day old rats), and those effecting the adult mature testis (in the rat this corresponds to greater than 40 days postnatal age). The ontogenical profile of Insl6 indicates that its expression in germ cells is significantly increased at 20 days of age and achieves maximum values at around 40 days of age. In view of these data, our efforts concentrated on putative effects of Insl6 on FSH's action on the Sertoli cell in the immature rat. FSH has pleiotropic actions on the Sertoli cell. The ability of Insl6, a peptide, to exert biological activity on the Sertoli cell is necessarily dependent on the presence of binding sites for Insl6 on the Sertoli cell. Insl6 exerts its biological function by specifically binding to its specific receptor protein.

The effects of Insl6 on the Sertoli cell comprise three actions of FSH: (1) FSH-stimulated induction of cyclic AMP (cAMP) production in Sertoli cells, (2) FSH-modulated metabolic activity of Sertoli cells, and (3) FSH-induced proliferation of Sertoli cells. Each is discussed in detail infra. FSH-stimulated cAMP production in Sertoli cells represents the canonical action of FSH on Sertoli cells and is the primary step in many of the actions of FSH on the Sertoli cell. A common feature of many of the prototypic members of the insulin gene family (e.g. insulin, insulin-like growth factors) is their ability to alter metabolism of target cells/organs. Data obtained with a mouse Sertoli cell line (MSC-1), support an inhibitory role for Insl6 in the proliferation of the Sertoli cell.

(1) FSH-Dependent Production of Cyclic-AMP—FSH binds to the cognate FSH receptor on the Sertoli cell to elicit robust increases in levels of intracellular cAMP. Insl6 has an effect on FSH-stimulated increase in cAMP production by Sertoli cells. Briefly, Sertoli cells from 15 day old rats were isolated as per the protocol described in the Experimental Methods section herein. The cells were maintained in serum-free medium and on the third day after isolation these cells were exposed to FSH (ovine FSH, National Hormone and Pituitary Program/NIDDK; 1-100 ng/ml) in the presence and absence of graded doses of recombinant Insl6. At various time points the cells were harvested and processed for estimation of acetylated cyclic AMP levels using an enzyme-linked immunoadsorbent (EIA) assay (LINCO). The sensitivity of this assay is in the picomolar range for acetylated cAMP.

(2)FSH-Dependent Lactate Production and y-Glutamyl Transpeptidase Activity—The distinctive functions of Sertoli cells are sustained by provision of adequate levels of energy substrates and protection against oxidative injury. FSH is an important regulator of many of the pathways involved in this homeostasis. Thus, lactate production and γ-glutamyl transpeptidase (γ-GTP) activity have been shown to be stimulated by FSH. There may be potential effects of Insl6 on FSH stimulated lactate and γ-GTP activity in cultures of primary Sertoli cells. Briefly, Sertoli cells were isolated from 17-day old rats as per the protocol outlined in the Experimental Methods set forth herein. The Sertoli cell cultures were maintained in serum-free medium for 3 days and then the medium replaced with serum-free medium supplemented with FSH (100 ng/ml) and graded doses of recombinant Insl6. 24 hours after exposure to Insl6 the medium were harvested for measurement of lactate and γ-GTP activity. Lactate concentration was measured by a standard method known by those skilled in the art involving conversion of NAD⁺ to NADH using a commercial kit (Sigma). γ-GTP was assayed by the calorimetric method based on using L-γ-glutamyl p-nitroanilide as substrate and glycylglycine as the acceptor molecule. Values were compared against a standard curve obtained with increasing concentrations of p-nitroaniline.

(3) FSH-Dependent Proliferation—FSH is the major regulator of adult Sertoli cell number because it stimulates the proliferation of Sertoli cells in the course of fetal and perinatal development. Furthermore the direct actions of FSH on the Sertoli cell can be either potentiated or inhibited indirectly by factors produced locally under the influence of FSH. For example, the direct stimulatory actions of FSH on the proliferation of immature Sertoli cell are potentiated by FSH-stimulated production of basic fibroblast growth factor (bFGF) by the Sertoli cell. However, at present, many aspects of the local modulation of FSH's actions on the Sertoli cell remain poorly understood especially in the context of putative inhibition of Sertoli cell proliferation by FSH during maturation from peripubertal to postpubertal development. Our results indicate that Insl6 inhibits the FSH-independent proliferation of transformed Sertoli (MSC-1) cells. Based on these data, it was found that one of the in vivo roles of Insl6 is inhibition of the proliferative effects of FSH on the Sertoli cell. This was determined by investigating the effects of recombinant Insl6 on FSH-stimulated proliferation of primary Sertoli cells. Briefly, rat primary Sertoli cell from 5 day old rats was isolated as per the protocol outlined in the Experimental Methods section below. Immature (5 day old) rats were used for this assay since Sertoli cells isolated from rats of this age exhibit a FSH-stimulated proliferative response. The cells were maintained in serum-free medium and on the third day after isolation these cells will be exposed to FSH (ovine FSH, National Hormone and Pituitary Program/NIDDK; 1-100 ng/ml) in the presence and absence of graded doses of recombinant Insl6. At selected time points the cells were harvested and the number of viable cells determined by a non-radioactive tetrazolium compound-based colorimetric assay (CellTiter; Promega); it is known by those skilled in the art that tetrazolium reagents can be substituted for the [³H]thymidine in the conventional assay.

Effects of Insl6 on Synthesis of Sertoli Cell Proteins

It is well established that proper functioning of the testis involves integration of different stimuli including hormones, locally secreted growth factors, cytokines, and adhesion molecules acting on the various cellular components of the testis such as the Sertoli, germ, and Leydig cells. Sertoli cells secrete a wide variety of proteins that play a role in maintenance of the intra-tubular and intercellular microenvironment in which meiotic and post-meiotic germ cells are sequestered. The characterized proteins secreted by the Sertoli cells can be grouped into (a) molecules involved in transport and bioprotection such as transferrin, ceruloplasmin and IGF binding proteins, (b) molecules with protease and protease inhibitor properties such as plasminogen activator and metalloproteinase, (c) components of the basement membrane such as collagen and laminin, and (d) hormone and growth factors such as IGF-1, inhibin, basic fibroblast growth factor, and mullerian inhibitory factor.

Data of the present invention indicate that Insl6 alters the tyrosine phosphorylation status of the Sertoli cell. The following are Sertoli cell proteins affected by Insl6: transferrin, inhibin, androgen binding protein (ABP), and IGF-1 and IGF-binding proteins, each discussed infra.

Transferrin—Transferrin is a major transporter of iron into cells and one of the most important elements required for growth and viability of all living systems. Transferrin is a major Sertoli cell glycoprotein product, and the rate of its synthesis is under complex control involving hormones (FSH and testosterone), retinoids, cytokines, and the surrounding germ cells. Previous studies have implicated TNF-α and basic fibroblast growth factor as factors that are secreted by germ cells that influence transferrin synthesis by Sertoli cells. Insl6 also plays a role in germ cell modulation of transferrin synthesis by Sertoli cells. This effect has been determined by studying the effect of Insl6 on production of transferrin by primary Sertoli cells. Briefly, Sertoli cells were isolated from 17 day old Sprague-Dawley rats as per the protocol outlined in the Experimental Methods section set forth herein. These cells were exposed to either graded doses of recombinant Insl6 or vehicle. After varying period (12-48 hours) of exposure to the conditioned medium the cells were harvested for extraction of RNA and the supernatant collected for estimation of transferrin content by radioimmunoblot using a polyclonal antibody raised against rat transferrin (Cappel Laboratories, Cochranville, PA). The extracted RNA was processed for measurement of transferrin mRNA by Northern blot analysis.

It is known by those skilled in the art that under the appropriate culture conditions transferrin synthesis by mature Sertoli cells is responsive to FSH. However the magnitude of FSH's stimulation of transferrin synthesis even under the appropriate culture conditions is only about two-fold. Hence it was chosen to initially test for effects of Insl6 on basal production of transferrin by Sertoli cells and considered investigating effects of Insl6 on FSH-stimulated transferrin synthesis if there is a marked effect of Insl6 on basal transferrin synthesis.

Inhibin—Inhibin is a product of Sertoli cells with significant physiological functions. There are two bioactive forms of inhibin: inhibin A and inhibin B. inhibin B is the physiologically important form in males. Prior studies have suggested that germ cells modulate the ability of Sertoli cells to synthesize inhibin. Thus in the rodent, Sertoli cell cultures lacking germ cells secrete higher levels of inhibin than those from comparable ages in which germ cells are present. Similarly, serum inhibin B concentrations are significantly higher in fertile controls than in men presenting with primary germ cell failure. Other investigators have demonstrated that inhibin B levels are correlated with both sperm count and testicular volume. At present, the germ cell factor(s) that regulate production of inhibin by the Sertoli cell are not known. Insl6 influences inhibin expression in Sertoli cells. For these experiments, rat primary Sertoli cells were isolated from 18 day old rats were exposed for varying time periods to either recombinant Insl6 (present in the conditioned media of the CHO stable cell line expressing Insl6) or conditioned medium from untransfected CHO cells. Following the exposure to Insl6 or control medium, RNA was extracted from these cells and subjected to Northern analysis for inhibin B. The abundance of the mRNA for a housekeeping gene, such as GAPDH, was estimated for the purpose of normalization of sample loading and transfer efficiency. Previous studies have demonstrated the feasibility of monitoring Sertoli cell function by measuring inhibin B production and accumulation in culture medium of rat primary Sertoli cells. Inhibin B was measured using the inhibin-B dimer assay kit (Serotex). This assay is a double antibody enzyme-immunometric assay that uses a monoclonal antibody raised against the inhibin [3B subunit in combination with an antibody directed against the inhibin βB subunit. Using this ELISA system we will confirm that Insl6 mediated alterations in inhibin B MRNA levels of rat primary Sertoli cells are accompanied by corresponding changes in the synthesis rate of inhibin B protein.

The question of modulation of inhibin B synthesis by FSH in isolated rat primary Sertoli cells is controversial. Whereas many studies have failed to demonstrate an effect of FSH on regulation of inhibin B levels in vitro, a recent study did demonstrate that FSH stimulated the release of inhibin B into the culture medium of isolated rat Sertoli cells. The difference in these results can be attributed to culture conditions including the inclusion of EGF and insulin in the culture medium and the temperature at which the cells were cultured. In addition to investigating the putative effects of Insl6 on basal production of inhibin B by isolated rat primary Sertoli cells, effects of Ins16 on FSH-stimulated inhibin B production were investigated using a similar model system. Briefly, rat primary Sertoli cells from 17 day old rats were isolated and enriched as per the standard procedure and then maintained at 34° C. in DMEM:HF-12 (1:1) medium supplemented with insulin (2 μg/ml), transferrin (5 μg/ml), and EGF (10 ng/ml). The effect of graded doses of recombinant Insl6 on the ability of FSH (ovine FSH, National Hormone and Pituitary Program/NIDDK) to stimulate inhibin B in a dose (1-100 ng/ml) and time-dependent (24-72 hrs) manner is assessed by assaying the culture medium for inhibin B using the above described ELISA assay.

Androgen Binding Protein (ABP)—ABP is a major secretory product of the Sertoli cell and is an important modifier of the action of androgens in the testis. ABP synthesis by the Sertoli cell is primarily under the control of FSH and androgens. Prior studies have also suggested a role for germ cells in the stimulating ABP secretion by the Sertoli cell. However, the factor(s) that may mediate this effect of germ cells on Sertoli cell synthesis of ABP are (is) unknown. Insl6 influences the synthesis of ABP by the Sertoli cell. Briefly, rat primary Sertoli cells from 17 day old rats were isolated and enriched as per the standard procedure and then maintained at 34° C. in DMEM:HF-12 (1:1) medium supplemented with insulin (2 ug/ml), transferrin (5 ug/ml), and EGF (10 ng/ml). The effect of graded doses of recombinant Insl6 on the ability of FSH (ovine FSH, National Hormone and Pituitary Program INIDDK) to stimulate ABP synthesis in a dose (1-100 ng/ml) and time-dependent (24-72 hrs) manner were assessed by assaying the culture medium for ABP using an R1A and by Northern blot analysis of the extracted RNA for abundance of ABP MRNA.

Under the appropriate culture conditions, ABP synthesis by mature Sertoli cells is responsive to FSH. However, the magnitude of FSH's stimulation of ABP synthesis even under the appropriate culture conditions is only about two-fold. Hence, it was chosen to test for effects of Insl6 on basal production of ABP by Sertoli cells and the effects of Insl6 on FSH-stimulated ABP synthesis.

IGF-1 and IGF-Binding Proteins (IGFBPs)—The IGFs, their receptors, and IGFBPs are cellular modulators that play essential roles in the regulation of growth and development. The biological roles of IGFs are modulated by a family of at least six IGF-binding proteins that are found in circulation and in the extracellular compartment. The IGFBPs can inhibit or enhance the effects of IGF and may also have ligand-independent effects. The Sertoli cell is a site of IGF-1 production. In general, the local production of IGF-1 in a particular tissue is under control of complex series of factors. In the Sertoli cell, IGF-1 production is stimulated by FSH, GH, and FGF. The IGF-1 synthesized by the Sertoli cell is believed to exert its effects on Sertoli and germ cells via autocrine and paracrine actions respectively. Thus IGF-1 stimulates proliferation of immature rat Sertoli cells and spermatogonial DNA synthesis, exerts a maintaining effect on premeiotic DNA synthesis, and in vitro induces the differentiation of type A spermatogonia. Both ligand blot analysis and mRNA analysis reveal that the predominant IGFBP synthesized by the Sertoli cell is IGFBP-3.

Insl6 regulates the IGF-1/IGFBP axis in Sertoli cells and effects on the IGF and IGFBP3 synthesis by Sertoli cells. Briefly, primary rat Sertoli cells were exposed for varying time periods to either recombinant Insl6 (present in the conditioned media of the CHO stable cell line expressing Insl6) or conditioned medium from untransfected CHO cells. Following the exposure to Insl6 or control medium, RNA was extracted from these cells and abundance of IGF-1 mRNA measured by a TaqMan assay previously established in our laboratory. The abundance of the mRNA for a housekeeping gene, such as GAPDH, was estimated for the purpose of normalization of input RNA. IGFBP3 was measured in the culture medium by an ELISA standardized for detecting rodent IGFBP3 (Diagnostic Systems Lab).

The Sertoli cells make a large number of proteins and it was not feasible to test for effects of Insl6 on synthesis of all known Sertoli cell proteins. The choice of the proteins enumerated above is primarily based on previous studies that suggest circumstantial links between germ cells and synthesis of the particular protein by the Sertoli cell. Similarly, it was chosen not to investigate some other major proteins synthesized by the Sertoli cell. For example clusterin (also termed apolipoprotein J/sulfated glycoprotein-2) is major product of the Sertoli cell. However previous data indicate that clusterin's synthesis by the Sertoli cell is not modulated by germ cells. Thus, co-culture of germ cells with rat Sertoli cells did not alter the abundance of clusterin mRMA levels in Sertoli celts. Furthermore, clusterin levels in Sertoli cell of adult rats that were irradiated in utero and hence devoid of germ cells were no different from that in control rats. On the basis of these data we determined that it is unlikely that Insl6 influences the synthesis of clusterin by the Sertoli cell

The choice of whether to test for effects of Insl6 on Sertoli cells under basal or FSH-stimulated conditions was necessarily an empirical decision. This is because whether an individual protein or metabolite synthesized/secreted by the Sertoli cell is influenced by FSH or not may be a reflection of the particular experimental condition, e.g. in vitro vs. in vivo and culture conditions. As noted above, this fact is exemplified by FSH stimulation of transferrin and ABP synthesis by the Sertoli cell

Although the experimental model of Sertoli cells in culture is routinely used to study the function of Sertoli cells, there are potential drawbacks in using this model for endocrine and paracrine studies. Thus, the magnitude of response of a given parameter to FSH can be dramatically affected by cell culture conditions. Factors such as culture medium, plating density of cells, incubation temperature, and time of addition of hormones all affect the response. Taking cognizance of these observations we strove to minimize these variables by conducting control and experimental perturbations under similar conditions.

Modulation-of Canonical Signaling Pathways in Sertoli Cells by Insl6

FIG. 11 shows results indicating that Insl6 alters the tyrosine phosphorylation status of a Sertoli cell line (MSC-I) proteins. Although Sertoli cell lines are useful tools for investigating Sertoli cell function, they are important differences between primary Sertoli cells and established Sertoli cell lines. For example, compared to primary Sertoli cells, MSC-1 cells are relatively deficient in the FSH receptor. Hence, to verify that the results obtained with the MSC-1 cells are representative of the effects of Insl6 in vivo, we confirmed these results using primary rat Sertoli cells. For this purpose, we exposed serum-deprived rat primary Sertoli cells to recombinant Insl6 in a serum-free milieu and determined the tyrosine phosphorylation profile of cellular proteins by Western blotting. Upon confirmation that the Insl6 alters the tyrosine phosphorylation status of cellular proteins in these primary Sertoli cells, we proceeded with determining the identity of the pathways involved in intra-cellular signaling by Insl6 in Sertoli cells. Based on the premise that Insl6 belongs to the insulin family of proteins, the following canonical signaling pathways involving Insl6 action on Sertoli cells were investigated:

Mitogen Activated Protein (MAP) Kinase—In mammals, MAPK signaling cascades regulate cellular processes including gene expression, cell proliferation, cell survival and death, and cell motility. Mammals express at least four distinctly regulated groups of MAPs, ERK1/2, JNK 1/2/3, p38 proteins (p38ot/B/γ/8), and ERK5. Each of these enzymes are activated by specific MAPK kinases (MAPKK): MEK1/2 for ERK1/2, MKK3/6 for p38, MKK4/7 (JNKK1/2) for the JNKs. Generally, activation of ERKI/2 is linked to cell survival, whereas JNK and p38 are linked to induction of apoptosis. Our results indicate that one of the protein(s) that displays increased tyrosine phosphorylation in response to Insl6 has a molecular weight that is compatible with p38 proteins (FIG. 11). The involvement of the MAP kinase pathway in Insl6 action in Sertoli cells by Western blotting using state-specific antibodies was investigated. Briefly, rat primary Sertoli cells will be serum-deprived for 24 hours and the exposed to Insl6 containing conditioned medium (from CHO cells stably expressing Insl6) for varying time periods. Cell lysates from these cells were size-fractionated by SDS-PAGE, transferred onto nitrocellulose membranes and probed with phosphorylation state-specific antibodies against ERK1/ERK2, p38, and JNK individually. Following autoradiography, the blots were stripped and re-blotted with an antibody that recognizes both the phosphorylated and non-phosphorylated forms of the individual MAP kinases.

Insulin Receptor Substrates—Insulin receptor substrates (IRS-1, IRS-2, and IRS-3) are a family of docking proteins that play key roles in signal transduction of the insulin receptor. IRS proteins are also involved in the signal transduction pathways of a wide variety of cytokines and other related growth factors such as IGF-I and growth hormone. It was investigated whether Insl6's actions on Sertoli cells involve IRS proteins. Briefly, rat primary Sertoli cells were serum-deprived for 24 hours and the exposed to different doses of recombinant Insl6 in a serum-free milieu for varying time periods. Cell lysates from these cells were immunoprecipitated with the respective anti-IRS antibody and the precipitated proteins size-fractionated by SDS-PAGE. Following transfer of the proteins onto nitrocellulose membranes the proteins will be probed with anti-phosphotyrosine antibody using the Western blot technique outlined in the Experimental Methods set forth herein.

Protein Kinase B (Akt)—The serine/threonine kinase protein kinase B (PKB/Akt) has been shown to play a crucial role in the control of diverse and important cellular functions such as cell survival and glycogen metabolism. There is also convincing evidence that PKB plays a role in the insulin-mediated regulation of glucose transport. Insulin has been shown to directly influence glucose uptake by cultured Sertoli cells. In contrast FSH influences Sertoli cell metabolism of glucose by stimulating the activity or levels of glycolytic enzymes. The Akt kinase exists as three different isoforms, all of which are activated by phosphorylation on residues T308 and S473. Upon growth factor stimulation, Akt localizes near the plasma membrane, where it becomes phosphorylated. The activated enzyme has the ability to translocate to the nucleus. Insl6's actions on the Sertoli cell involve activation of Akt. For these experiments rat primary Sertoli cells were serum-deprived for 24 hours and then exposed to different doses of recombinant Insl6 for varying time periods. Cell lysates from these cells were size-fractionated by SDS-PAGE, transferred onto nitrocellulose membranes and probed with phosphorylation state-specific antibodies against Akt. Following autoradiography, the blots will be stripped and re-blotted with an antibody that recognizes both the phosphorylated and non-phosphorylated forms of Akt.

### EXPERIMENTAL METHODS

Isolation and culture of primary rat Sertoli cells—Rat Sertoli cells were isolated in our laboratory using a standardized protocol. Briefly, testes from Sprague-Dawley rats of the indicated age were harvested, decapsulated, and digested with collagenase (0.5 mg/ml, 33° C., 12 rain) in enriched Krebs-Ringer bicarbonate media (EKRB), followed by three washes in EKRB (1×g, 3 rain). The seminiferous tubules isolated were then digested with trypsin (0.5 mg/ml, 33° C., 12 min), and cell aggregates passed repeatedly through a drawn-out Pasteur pipette. An equal volume of DMEM containing 10% FCS will be added to the Sertoli cells, which were then pelleted (500×g, 5 min) and resuspended in serum-free media containing 50% DMEM, 50% Ham's F12, 5 μg/ml insulin, 5 μg/ml transferrin, 10-6 M retinoic acid, 10 ng/ml epidermal growth factor, 3 μg/ml cytosine B-D-arabinofurano-sidase, 2 mM glutamine, 1 mM sodium pyruvate, 100 U/ml penicillin, and 100 μg/ml streptomycin. Sertoli cells were cultured on matrigel (Collaborative Research, Bedford, Mass.)-coated dishes (32° C., 5% CO2) and routinely used for experimentation 72 hrs following the isolation procedure. The purity of the Sertoli cell isolate yielded by this protocol is >95% as determined by phase microscopy and alkaline phosphatase staining.

Western Blot Analysis—Western blot analyses were carried out by established protocols known by those skilled in art. Briefly, following the experimental perturbation cells will be harvested by first washing in cold 1×PBS, then scraped into cold lysis buffer (20 mM HEPES, pH 7.4, 100 mM NaCI, 4 mM sodium pyrophosphate, 2 mM sodium orthovanadate, 10 mM EDTA, 1 mM phenylmethylsulfonyl fluoride (PMSF), 10 mM NaF, 1% (v/v) Triton X-100, 2 μg/ml leupeptin, and 2 μg/ml aprotinin). After centrifugation at 15,000×g for 15 min at 4° C., 10-15 microgram aliquots of the detergent extracts will be resolved under reduced conditions by SDS-PAGE prior to transfer to nitrocellulose for probing with the respective primary antibody. Equality of loading and efficiency of transfer were assessed by Ponceau staining of the nitrocellulose filters and protein concentration determined by Bradford's method (BioRad). Detection of the primary antibody will be with horseradish peroxidase-conjugated goat anti-mouse antibody using a chemiluminescent system (Amersham, Arlington Height, Ill.).

Antibodies—Anti-phosphotyrosine monoclonal antibody (4G10) and anti-MAPK affinity-purified rabbit antibody (directed at residues 333-367 of rat ERKI; recognizes both ERK1 and ERK2) was purchased from Upstate Biotechnology, Inc., Lake Placid, N.Y. (UBI). Anti-activated (anti-phospho-) MAPK affinity-purified rabbit antibody (recognizing the dually phosphorylated Thr183 and Tyr185 residues that correspond to the active forms of ERK1 and ERK2) is available from Promega, Inc. (Madison, Wis.). Anti-Akt and anti-phospho-Akt (specifically recognizing Akt phosphorylated at Ser473) affinity purified rabbit antibodies were obtained from New England Biolabs (Beverly, Mass.).

Targeted Disruption of the Murine Insl6 Gene

Targeted disruption of the Insl6 gene in the mouse model revealed the biological role(s) of Ins16. Insl6 is evolutionarily conserved. Expression of Insl6 displays a tissue-specific pattern of expression with expression being most abundant in spermatocytes. One of the strategies to further define functional role(s) of Insl6 is to create a null phenotype by targeted deletion of the gene in the mouse. The targeted disruption of Insl6 gene was achieved by a strategy employing homologous recombination. The salient steps in this process are as follows: (1) Screening of a 129/Sv genomic library (xFix; Stratagene) to isolate lambda clone(s) containing the Insl6 gene. (2) Mapping intron/exon boundaries using a combination of DNA sequencing, restriction mapping, and PCR-based exon trapping strategies. Based on our mapping of the human INSL6 gene, it was anticipated that the murine Insl6 gene contains two exons. Since there was no a priori evidence of any functional domains within the Insl6 gene, these studies were initiated by targeting the first exon. (3) Constructing a target vector containing HSV-TK and neomycin resistance genes flanked by LoxP sites (LTNL cassette), placed in transcriptional orientation opposite to that of the first exon of the Insl6 gene. This allows for positive/negative selection and deletion of exon 1 of Insl6 and insertion of the LINL cassette. The vector backbone was provided by Dr. R. Chaillet, Children's Hospital of Pittsburgh. (4) Intoducing linearized targeting vector DNA by electroporation into 129/Sv ES cells and, selection of recombinant clones achieved by growth in the presence of G418 (400/gms/ml). The clones were checked for recombination using Southern blot analysis, the LTNL cassette excised by transient transfection of a Cre-expressing plasmid (e.g. CMV-Cre), and putative clones selected by growth in the presence of gancyclovir (2 μM). (5) Generating chimeras by injecting cells of the identified recombinant ES clone into host blastocyst of C57B/6J mice with subsequent transfer into uteri of pseudo-pregnant females. (6) Mating of male chimeras with C57BL/6J females and Fl heterozygous (Insl6^{-/+}) progeny were intercrossed to obtain Insl6 null (Insl6^{-/-}) mice.

The absence of the Insl6 transcript was assessed by Northern blot analysis and confirmed by TaqMan assays. The RNA analyzed was from tissues where Insl6 is expressed such as testis, small bowel, and kidney. The lack of Insl6 protein in these tissues will be confirmed by Western blotting. The phenotype of the Insl6^{-/-}) mice was initially examined by comparing the following characteristics with those of the wild type mice:

(1) Viability—Insl6 expression is expressed only postnatally in the site of greatest expression, i.e. testis. Hence, encountering embryonic lethality or increased perinatal mortality/morbidity in the Insl6 null (Insl6′-) mice.

(2) Fertility—Based on the expression pattern of Insl6, the effect of Insl6 ablation on fertility is more pronounced in males than in females. However, since Insl6 mRNA expression can be detected in uterus and ovary, abnormalities in fertility in the female Insl6 null (Insl6^{-/-}) mice were carefully examined. For this analysis the consequence of Insl6 disruption on fertility will be compared between heterozygote intercrosses and reciprocal crosses of heterozygous mice to homozygous targeted mice. To ensure genetic identity, only F1 progeny heterozygous mice were used for this evaluation of fertility. The number of productive matings and the average first litter size of all matings was tabulated. A decrease in fertility rate (percent of productive matings) and/or litter size for either male or female homozygous mutant mice suggests an effect of Insl6 null (Insl6^{-/-}) phenotype on fertility.

(3) Testicular Macro and Microstructure—Both gross macroscopic features (size and weight) as well as microscopic features of t-13 testis from the adult animal were surveyed to discern changes in architecture and organization of the testis in Insl6 null (Insl6-) mice. Thus the histological characteristics that were specifically examined for include diameter and organization of the seminiferous epithelium, relative numbers of spermatogonia, round and elongated spermatids.

(4) Sperm Motility Analysis—Our data show that a mutation in the human INSL6 gene causes decreased sperm motility resulting in male infertility. PCR was used to amplify the exon 1 fragment of the human INLS6 gene encompassing a putative mutation identified by Single Strand Conformational Polymorphism (SCCP) analysis in patient #2. The PCR product was subjected to restriction analysis with BsmF I restriction endonuclease. Wild type sequence (patients # 1, 3, 4) predicted bands of 300 and 60 (not shown) bp; the heterozygous 22 to A nucleotide change in patient #2 predicts loss of the BsmF I site and appearance of an additional 360 bp band. Briefly, spermatozoa were collected from cauda epididymis and capacitated for 60 minutes at 37° C. in 5% CO₂/5% O₂/90% air. Motility parameters were measured using a computer-assisted digital analysis system as known by those skilled in the art. The following parameters will be measured: velocity (curvilinear velocity, μm/sec, the total distance divided by time), linearity (the distance in straight line divided by actual distance), amplitude of lateral head displacement (ALH, μm, the deviation of the head from the mean head trajectory), ALHₘₐₓ (the maximal ALH observed for each spermatozoa), beat cross frequency (beats per second, the number of times that the spermatozoa head crosses the mean head trajectory per second), percent of circular cell (number of spermatozoa swimming in circles divided by the total number of motile spermatozoa), hyperactivation (the number of spermatozoa with a velocity greater than 136 μm per sec, a linearity <5.5 and a ALHₐᵥₑ>8 μm divided by the total number of motile spermatozoa).

(5) Endocrine Profile—Insl6 may have biological effects acting as a circulating endocrine factor. The Insl6 null model was analyzed for evidence to support this. Thus we measured circulating random levels of LH, FSH, and testosterone.

Insl6 is a single copy gene and hence, we did not anticipate difficulties in cloning of the requisite stretches of genomic DNA. The lack of an obvious phenotypic alteration in the Insl6 null (Insl6^{-/-}) mice would suggest redundancy in the biological role of Insl6. However before such a conclusion was reached we explored in detail the phenotype of the Insl6^{-/-} mice with special emphasis on possible alteration in phenotype related to sites of expression of the Insl6 other than the gonads. Hence gastrointestinal function was assessed for evidence of malabsorption and renal functions for evidence of abnormalities in glomerular and tubular functions.

Mutations in the INSL6 Gene in Patient Populations

Based on the expression pattern of 1NSL 6, we determined that loss-of-function mutations were identified in men with impaired sperm motility. Our results indicate that Insl6 is expressed in spermatocytes, intestine, kidney and vomero-nasal epithelium. This spectrum of expression led to demonstrating that Insl6 plays a role in motility. Given that the expression of Insl6 is maximal in spermatocytes, it was reasoned that one of the phenotypes of a loss of function mutation of the Insl6 gene in the human could be infertility characterized by impaired sperm motility. Studies identified a missense mutation in the Insl6 gene in a subject with male infertility and impaired sperm motility. The details of the investigation designed to identify and study the role of variations in the nucleotide sequence of the Insl6 gene in the human is described below.

Blood samples were collected from men with infertility. The blood samples were extracted for genomic DNA from peripheral blood lymphocytes. Clinical information regarding fertility, history of cryptorchidism, sperm count, sperm motility, and sperm morphology were analyzed.

Genomic analysis: SSCP (single strand conformational polymorphism) analysis was utilized to screen for mutations in INSL6. For this purpose SSCP analysis of each exon including intron/exon boundary regions was performed. In this method, alleles were PCR amplified in the presence of ³²P-dATP and segregated by differing electrophoretic mobilities. When denatured and electrophoresed in a non-denaturing (native) polyacrylamide gel matrix, each single DNA strand (conformer) is unable to hybridize with its complementary strand and, instead, reanneals within itself to achieve its lowest free energy form. Two complementary DNA strands are generally visualized as two distinct bands with characteristic mobility patterns on autoradiography. The specific conformer for each DNA strand is usually exquisitely sequence dependent such that a single nucleotide difference is easily detected. In DNA amplicons of 200-500 basepairs, single nucleotide changes generally alter the lowest free energy state. Hence, in a subject heterozygous for a point mutation, the mobility of the four complementary single DNA strands differs so that four bands would be anticipated on the autoradiograph. It is important to note that one DNA strand may exhibit different free energy states and may appear as multiple bands on autoradiography.

Upon detection of a conformer, the respective band(s) was excised from the dried gels and eluted into 100 μl 0.1×TE. A 5 μl aliquot of each eluted conformer was re-amplified using the original primer pair and thermocycling conditions. The amplicons were sequenced either manually using the Sequitherm Excel II DNA sequencing kit (Epicentre Technologies, Madison, Wis.) or using an ABI 310 automated sequencer (PE Applied Biosystems, Foster City, Calif.) according to the manufacturer's protocol. Nucleotide sequences were compared to the wild type and variants noted. To verify the presence of a variant, all amplicons were sequenced using both sense and anti-sense primers. Next, it was determined if the variant alters a restriction site. If so, PCR and restriction fragment digestion derived from the original genomic DNA sample was performed to confirm the presence of the sequence variant.

For INSL6, SSCP analysis for 100 subjects with impaired sperm motility was performed. When a sequence variant was identified, SSCP analysis for 100 control subjects was performed. If a conformer is identified in both patients and controls in equal frequencies, then it is deemed to be a polymorphism. The conformer is then sequenced, but no additional DNA samples are analyzed. If the frequency of the conformer is greater among the subjects than the controls, additional male subjects with infertility are then analyzed by SSCP analysis to determine the frequency of the specific variant. To be considered a deleterious mutation, the base change must cause a missense or nonsense mutation, alter conserved splicing or promoter region response elements, occur in less than 1% of a normal population, and co-segregate with the phenotype in a family. Large deletions can be detected by apparent homozygosity for multiple polymorphisms or Southern blot analysis.

To date, we have identified a sequence variant in ⅕ genomic DNA samples evaluated by SSCP analysis. Hence, it is anticipated to detect other variants. The major limitation of SSCP analysis is the possibility of false negative results. To minimize the potential for false negative results, it was attempted to keep amplicon size between 150-450 bp because the sensitivity of SSCP for nucleotide changes is greatest with smaller PCR fragments. To further minimize the possibility of false negative results, three distinct gel conditions are utilized: 1) MDE gel (BioWhittaker Molecular Applications, Rockland, Me.) at room temperature for 12-16 hours; 2) 5% polyacrylamide at 4° C. for 3-6 hours; and 3) 5% polyacrylamide with 5% glycerol at room temperature for 5-8 hours. Duration of electrophoresis was chosen on the basis of amplicon size and adjusted as necessary so that the conformers are positioned in the mid-to-bottom third of the gel. Using these three different conditions, approximately 85-90% of sequence variants are routinely detected.

Following detection of a sequence variant, the functional consequence of each identified mutation is assessed. Thus nonsense mutations provided proof of the production of truncated proteins and may not require further investigation especially in the homozygous state. In contrast missense mutations require further analysis to discern whether the mutation is a silent mutation without alteration in the phenotype or it is responsible for the altered phenotype, i.e. infertility due to decreased sperm motility. For this purpose, the mutated protein was expressed in insect cells to test the biological effects of the expressed protein. The effect of Insl6 on proliferation of MSC-1 cells may be used as a biological assay.

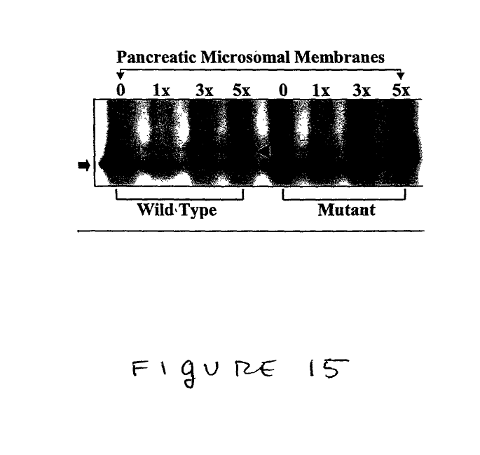

Mutations in the signal peptide of various genes have been demonstrated to alter trafficking of the respective protein or to effect the rate of translation of the protein. Computer analysis of the predicted primary/secondary structure indicated that mutation in codon 8 of INSL6 would not result in alteration in cleavage of the signal peptide. To investigate the effect of this mutation on translocation and translation of INSL6, the impact of the mutation on INSL6 synthesis was studied using an in vitro TNT/T7 quick-coupled transcription/translation system (Promega) in the presence and absence of canine pancreatic microsomal membranes (Promega) which indicated that the mutant DNA exhibited a reduced efficiency of translocation in the endoplasmic reticulum (FIG. 15). Briefly, in vitro transcription and translation of the wild type and mutant templates were performed using reticulocyte lysates in the presence of [³⁵S] L-cysteine and with and without the addition of graded amounts, 1×, 3× or 5×, of canine pancreatic microsomal membranes. The reaction products were size fractionated via SDS-PAGE and autoradiographed. The mutant INSL6 remains untranslocated species (as shown by the arrow symbol in FIG. 15) while the wild type is observed in the translocated species (as shown by the diamond symbol in FIG. 15). These results suggest a molecular basis for an abnormality in INSL6 synthesis and function. Further studies will concentrate on validating these results by determining the orientation of protein domains across the endoplasmic membrane by exploting the principle that the lipid bilayer of the microsomal membrane will protect the translocated (but not the untranslocated) protein domain from the proteolytic digestion.

The major limitation of SCCP is the possibility of false negative results. To minimize the potential for false negative results, amplicon size was limited to between 150-450 bp because the sensitivity of SCCP for single nucleotide changes is greatest with smaller PCR fragments. Another drawback of SCCP analysis as a diagnotic tool is the inability to detect large deletions because it may be difficult to distinguish homozygosity from loss of heterozygosity. To assure that large INSL6 gene deletions are not overlooked, microsatellite markers in the region of the INSL6 gene are utilized as a further diagnostic tool. Allele specific primers will be utilized. As these markers are sequence tagged sites from polymorphic regions, heterozygosity indicates the presence of tow alleles. If a subject is homozygous for all markers, Southern blot analysis is performed to confirm the presence of a large gene deletion. Specific markers utilized are as listed in Table I below.

TABLE I

| Location | Marker | Category |
|---|---|---|
| 9p21.1 | D9S251 (UT2100) | Microsatellite repeat |
| 9p21.1 | D9S248 (UT5132) | Tetranucleotide repeat |
| 9p23 | D9S256 (AFM161xd6) | Dinucleotide repeat |
| 9p24.1 | D9S1686 (AFM238zcl) | Dinucleotide repeat |
| 9p24.1 | D9S1873 (AFM362tdl) | Dinucleotide repeat |
| 9p24.1 | D9S1792 (AFMa184wh5) | Dinucleotide repeat |
| 9p24.3 | D9S1858 (AFM274xe1) | Dinucleotide repeat |

Expression of Insl6 is Regulated by Cyclic AMP and Hormonal Factors.

Our results indicate dramatic ontogenic changes in the abundance of Insl6 mRNA (FIG. 9). Whereas there is yet no direct proof that increased transcription rate (versus other mechanisms such as alteration in RNA stability) is responsible for these ontogenic changes in Insl6 mRNA levels, the magnitude of these changes (e.g. >20-40 fold increase in Insl6 mRNA abundance peripubertally) makes it likely that alterations in Ins16 gene transcription play a significant role in regulation of Insl6 expression

In general, mechanisms involved in transduction of extracellular signals to alter gene transcription can be grouped into three categories: (a) phosphorylation cascades, in which successive kinases are activated by phosphorylation and or dephosphorylation; (b) proteolytic cascades in which active molecules are cleaved from inactive precursors; and (c) systems that rely on non-protein second messengers such as cyclic nucleotides, calcium, and phospholipids. Our preliminary data suggest a role for cyclic AMP (cAMP) in the regulation of expression of Insl6. The pathways involved in cAMP-mediated regulation of Insl6 gene expression were investigated. Expression of Insl6 in the testis is significantly increased at the time of pubertal maturation in the rodent (FIG. 8). Puberty is associated with profound changes in hormonal profile with significant alterations in hormones such as gonadotropins, androgens, growth factors including growth hormone and Igf-1, and anabolic hormones such as insulin. The role of various hormonal factors with potential roles in the regulation of expression of Insl6 was investigated. These studies compliment the investigation into the role of cAMP in modulating expression of Insl6.

The basic experimental paradigm was to study the effect of cyclic AMP and of hormones on the expression of Insl6 gene in primary germ cells in culture. Leads obtained by these in vitro experiments were then be followed by investigating in vivo models. This sequence of experiments with in vitro experiments preceding the in vivo experiments was dictated by the fact that the presence of feedback loops and cell-cell interactions in the intact testis could have vitiated the interpretation of in vivo data On the other hand in vitro experiments by themselves may not provide a complete picture of the biology or Insl6 expression since factors made by adjacent cells (e.g. Sertoli cell) may have an important role in determining the complete profile of expression of Insl6. For these experiments, in general, Insl6 expression has been thus far profiled by measuring Insl6 mRNA by TaqMan assay. In relevant instances upon demonstration of significant effect of any of the hormones on Insl6 mRNA expression, Western blot analyses confirm that there are corresponding changes in levels of Insl6 protein. Western blot analysis were carried out using polyclonal rabbit anti-peptide. Briefly, anti-Insl6 antibody was used in Western blot, under reducing conditions SDS-PAGE, of cell lysates of CHO cells stably expressing Insl6. FIG. 6B shows the specificity of the detected signal was ascertained by determination of the molecular weight and by competition with molar excess of immunizing peptide. These results indicate that the anti-Insl6 antiserum is sufficiently specific and of an adequate titer to perform western blotting and quantitation of Insl6 expression. This analysis indicates that antiserum of a reasonable titer had been obtained (FIG. 6B).

Second Messenger Systems

Data indicate that cAMP increases the expression of Insl6 in germ cells. Further studies further confirmed that these effects of cAMP on steady-state levels of Insl6 are the result of alterations in gene transcription. Gene transcription rate was estimated by transcription run-on assays. The run-off transcription assay is often used to assess whether changes in mRNA levels of a particular gene that occur as a function of cell state reflect a change in its synthesis, as opposed to a change in mRNA degradation or transport from the nucleus to the cytoplasm. Briefly, nuclei were isolated from primary germ cells exposed to forskolin/IBMX and then incubated with ³²P-labeled UTP and unlabeled NTPs to label nascent RNA transcripts. ³²P labeled RNA was purified and used to detect specific RNA transcripts by hybridization to Insl6 cDNA immobilized on nitrocellulose membranes. A variety of different nuclear runoff transcription protocols have been described. The procedures differ primarily in the method of isolation of ³²P-labeled RNA. We chose a protocol reported to be advantageous because of low levels of background transcription due in part to inclusion of a TCA precipitation step, which allows effective removal of unincorporated ³²P and limited digestion of ³²P-labeled RNA with NaOH to facilitate hybridization.

A critical step in the nuclear runoff assay is isolation of nuclei. Thus, poor incorporation of ³²P-labeled UTP into RNA in isolated nuclei may reflect damage to the nuclei during isolation or failure to isolate nuclei free of cytoplasmic and membrane contaminants. There was no a priori evidence that any particular protocol was uniquely suited for isolating nuclei from spermatocytes. Thus to ensure superior quality of spermatocyte nuclei isolated, parameters such as use of detergents (e.g. NP-40), addition of Dounce homogenization, and isolation of nuclei by sucrose gradient centrifugation were evaluated. Particular care was taken in these experiments to ensure that the same number of nuclei was used for each cell state analyzed. In addition, the overall level of RNA synthesis by measuring the transcription rate of a house-keeping gene such as GAPDH was monitored to ensure that observed alterations in synthesis rate of Insl6 were not non-specific effects.

Upon verification that the effects of cAMP on Insl6 expression are due to alterations in gene transcription it was investigated whether these effects require de novo protein synthesis by testing the effect of inhibitors of protein synthesis such as cyclohexamide on cAMP-stimulated Insl6 gene expression. Briefly, primary germ cells were exposed to forskolin/IBMX in the absence or presence of cyclohexamide (2-3 micrograms/ml) and the abundance of Insl6 mRNA estimated by TaqMan analysis. The effects of cAMP on gene transcription are believed to be primarily mediated by the protein kinase A (PKA) pathway. Alternate pathways involving activation of protein kinase B (PKB/Akt) and PI-3-Kinase also play roles in transducing cAMP's actions. It was determined which of these pathways is involved in cAMP regulation of Insl6 by testing effects of activators and inhibitors specific for each of these enzymes. 8-Bromo-cAMP is a cell-permeable cAMP analogue that activates protein kinase A and its effect on Insl6 expression was first tested to ascertain if PKA is involved in cAMP-mediated regulation of Insl6 expression.

Additionally, the effect of KT 5720 (Tocris) a potent, selective inhibitor of PKA (Ki=60 nM) with no effect on PKG or PKC (Ki>2 uM) was also tested to ascertain the role of PKA in cAMP mediated regulation of Insl6. The effect of TPA (25 nM) was ascertained to determine the role of PKC-dependent pathways on Insl6 expression. Selective inhibitors of the P1-3-kinase pathway (e.g. Wortmanin and LY294002) were used to determine if the effects of cAMP on Insl6 are mediated via PI-3-kinase pathway. In general the effects of these compounds were assessed by using the TaqMan assay to measure cAMP-mediated Insl6 mRNA expression in rat primary germ cells in the absence and presence of a particular compound.

Role of PACAP in Modulation of Insl6 Expression

Pituitary adenylate cyclase-activating polypeptide (PCAP) is a member of the vasoactive intestinal peptide/secretin/GH-releasing factor family of peptide. PACAP was originally isolated based on its property of stimulating adenylate cyclase activity in pituitary cells. Although PACAP was originally isolated from the hypothalamus, PACAP expression is abundant in the testis. Although the precise role of PACAP is not known, available data suggest that PACAP participates in the regulation of spermatogenesis. It has been postulated that PACAP plays a role in the in vivo regulation of expression of Insl6 in germ cells. This hypothesis is based on the following observations: (1) spatio-temporal co-localization of Insl6 and PACAP in testis. Thus in the testis, PACAP is expressed in spermatids but not in Sertoli cells or Leydig cells, a pattern similar to that of Insl6. Additionally, PACAP expression in the testis is first detected at postnatal day 22-23 a time period when Insl6 expression is also increasing in the testis (FIG. 8). (2) cAMP increases expression of Insl6 mRNA in rat primary germ cells (FIG. 9A). This hypothesis was tested by determining the effect of PACAP on Insl6 expression in rat primary germ cells in culture. Briefly, rat primary germ cells in culture were exposed to 20 nM PACAP (Calbiochem) for varying time periods and RNA isolated for measurement of Insl6 mRNA by TaqMan assay. The intracellular levels of cAMP was also monitored to ensure activity of PCACP in this experimental model. Acetylated cyclic AMP levels were measured by an enzyme-linked immunoadsorbent (EIA) assay (LINCO).

Hormonal Factors

There are two principal hormonal factors in Insl6 expression, a) the gonadotropins, and b) growth hormone.

a) Gonadotropins—Among the hormonal factors affecting Insl6 expression are the gonadotropins. Testicular function is regulated by pituitary gonadotropins and by local cellular interactions that are influenced by pituitary LH acting on Leydig cells and FSH acting on Sertoli cells. Leydig cells produce testosterone and other androgens that regulate both Sertoli and germ cell functions. Similarly, Sertoli cells produce a variety of factors including growth factors that influence germ cell function. Therefore, it is possible that gonadotropins actions on Sertoli and Leydig cells may indirectly affect Insl6 expression in germ cells.

The regulatory role of gonadotropins on Insl6 expression by examining the effects of GnRH antagonists on Insl6 expression, with or without replacement treatments with LH, FSH, LH+FSH, or testosterone is set forth herein. Briefly, male rats of the SD strain (200-250 gm; Hilltop Labs, Allison Park, PA) were administered GnRH antagonist (Cetrorelix100 mcg/day intraperitoneally) or corresponding volume of the vehicle as the control. These animals were maintained on standard Purina Lab Chow and tap water. The hormone replacement regimens included subcutaneous administration of testosterone (Sigma; 5 mg/Kg body weight. dissolved in corn oil and administered intraperitoneally), ovine LH (Sigma; 25 mg/d), porcine FSH (Sigma; 25 mg/d), ovine LH+porcine FSH or vehicle only. The animals were euthanized after various time periods (1, 3, 5 and 7 days) and one testis and epididymis from each animal processed for histological examination and the contralateral testis harvested for extraction of RNA and protein. Trunk blood will collected for measurement of hormones. Abundance of Insl6 mRNA and protein was measured by TaqMan and western blot assays respectively. Levels of LH, FSH, testosterone were measured by radioimmunoassay.

To exclude effects of short-term administration of GnRH antagonists on germ cells number and morphology, the testis were subjected to histological examination. Briefly, testicular and epididymal tissue was fixed overnight in Bouin's solution at room temperature. After fixation, the tissue was dehydrated, embedded in paraffin, sectioned, deparaffinized, and stained with periodic acid-Schiffs haemotoxylin. One histological section was chosen at random from each animal and coded before cell enumeration to facilitate an unbiased assessment. Germ and Sertoli cells were counted in 25 seminiferous tubule cross-sections containing stage VII cellular associations. Sertoli cells were identified by their characteristic nucleolar morphology and will be counted to control for tissue shrinkage. Stage VII will be chosen because this stage contains all the necessary cell types to permit an assessment of the state of spermatogenesis. Cell numbers were corrected using the Abercombie formula well known by those skilled in the art.

The intimate functional and physical relationship between the Sertoli, Leydig, and germ cell is a crucial factor in the interpretation of data obtained by perturbation of individual components of this triad. Hence changes observed in the functioning of any one of these cell types upon administration of a hormone to an intact animal could be due to either a direct effect of the hormone per se or an indirect effect from altering the functioning of the other members of this triad. This conundrum is one reason for studying the regulation of function of germ cells in culture where the effects of the Sertoli and Leydig cells can be eliminated or controlled. Thus it was proposed to conduct our initial experiments regarding cAMP regulation of Insl6 expression using cultures of rat primary germ cells. However, the inter-relationship between Sertoli, Leydig and germ cells also necessitates that data obtained in vitro be confirmed in the milieu of the intact animal. Although in general the “hypox” (chemical or surgical) model is a accepted model for studying the effects of pituitary gonadotropins on gene expression, the fact that Insl6 is only expressed in late stage germ cells constraints the usefulness of this model for investigating the role of gonadotropins in modulating Insl6 expression. Hence it is not be possible to study the long term effects of hypophysectomy on Insl6 expression since prolonged deficiency of gonadotropins results in the atrophy of germ cells and consequent decrease in abundance of Ins16 mRNA in the testis. For this reason it was chosen to restrict the time course of the study to less than a week to minimize the possibility of germ cell atrophy. It was chosen to use GnRH antagonist rather than surgical hypohysectomy because of the likelihood of encountering more confounding variables in the surgical model. Thus the need to replace other pituitary hormones (such as thyroid and cortisol) and the possible effects of stress-induced cytokines and hormones released at the time of the surgical intervention made the use of GnRH antagonists a more “clean” model of gonadotropin deficiency.

One of the aims of using the GnRH antagonist model was to investigate the role of testosterone in regulating expression of Insl6. Thus observed effects of replacement of testosterone to animals administered GnRH antagonist may reflect the direct action of testosterone. However it should be noted that previous reports have indicated that administration of testosterone per se is capable of maintaining serum and pituitary FSH levels in vivo, under conditions which render the pituitary insensitive to hypothalamic GnRH. Whether this observed effect was unique to the type of GnRH antagonist (RS 68439) used in the particular study or the schedule of administration of testosterone is not clear. Nonetheless this caveat has been taken into consideration in interpreting the present results. The measurement of FSH in the various animals groups was considered and the effect of testosterone, if it plays a role in our experimental model, was discerned.

b) Growth Hormone

Our data in the rodent indicate that Insl6 expression significantly increases at around 20 days postnatal, coincident with onset of puberty. Growth hormone (GH) is one of the hormones whose secretion is increased markedly with onset of puberty. Additionally GH has been implicated in the regulation of male reproductive function. GH receptors are expressed on Leydig, Sertoli cells, spermatogonia, primary and secondary spermatocytes, and spermatids. Many of the actions of GH at the cellular level are believed to be mediated via generation of Igf-1. Igf-1 interacts with its cognate Igf-1 receptor to elicit actions including differentiation and proliferation. Igf-l receptors have been demonstrated on secondary spermatocytes and early spermatids, and to a lesser degree on Sertoli and Leydig cells. One model envisages that GH stimulates Igf-1 generation by Sertoli and Leydig cells and this signal is transduced to germ cells via the intimate association between Sertoli and germ cells.

Hence, the hypothesis that GH modulates the expression of Insl6 was tested by investigating the effects of GH administration on Insl6 gene expression in the testis. Two experimental models were employed: (a) administration of GH to a normal mice, and (b) replacement of GH in a model of chronic deficiency of GH. For the first model, C57BL male mice (4-6 weeks old) were treated with hGH (3 mg/kg twice a day for 5 days.). In the second model involving chronic GH deficiency {Lin S C, Lin C R, et al. 1993 ID: 954}, adult male mice homozygous for mutation in the GHRH gene (C57BL/6J-Ghrhr lit/+; Jackson Labs) mice were treated with a similar regimen. Respective control groups will receive vehicle only. At the end of the experimental period the animals were euthanized and testes harvested for extraction of RNA and protein.

The current dogma is that GH stimulates the local production of Igf-1 and that Igf-1 is the proximate mediator of GH's action in tissues. The actions of Igf-1 on cells are also influenced by IGF-binding proteins and in the conventional model IGFBP 1 antagonizes the actions of Igf-1 on tissues. Thus effects attributed to Igf-1 may in fact be the result of changes in IGF binding protein levels, which then influence Igf-1 action. To investigate this possibility it was also estimated by ligand blotting techniques the levels of IGF binding proteins in the testis of GH treated and control mice.

Experimental Methods

TaqMan Assay—Real-time quantitative RT-PCR using the ABI Prism 7700 Sequence Detection System (PE Biosystems; Foster City, Calif.) will be carried out using protocols standardized during generation of the data (FIGS. 7-9). The primers (synthesized by Life Technologies) and TaqMan probes (synthesized by PE Biosystems) for the quantitation of the murine and rat Insl6 transcripts were designed using the primer design software Primer Express (PE Biosystems: Foster City, Calif.). The primers and TaqMan probe for 18S rRNA were purchased from a commercial vendor (PE Biosystems; Foster City, Calif.). The 18S probe was labeled with reporter flourescent dyes VIC and the murine and rat INSL6 probes with FAM. The relative efficiencies of the INSL6 primers/probe sets and the 18S primer/probe pair were tested by subjecting serial dilutions of a single RNA sample from each of the tissues analyzed to real-time RT-PCR analysis. The plot of log input versus ACT was <0.1, which satisfies the previously established criterion for equivalence of efficiency of amplification {PE Applied Biosystems 1997 ID: 838}. CT or threshold cycle, represents the PCR cycle at which an increase in reporter fluorescence above a baseline signal can first be detected and ACT refers to the difference between the threshold cycles for the target and the reference. After confirming that the efficiency of amplification of the INSL6 gene and 18S transcripts were approximately equal, the amount of the transcripts for the specific gene relative to the 18S transcript was determined by using the comparative C_{T} (separate tube) method {PE Applied Biosystems 1997 ID: 838}. The comparative C_{T} method is similar to the conventional standard curve method, except it uses arithmetic formulas to achieve the same result for relative quantitation. The amount of target, normalized to an endogenous reference and relative to a calibrator is given by the formula: Fold Induction=2^{-ΔΔC} where ^{ΔΔC}T=[C_{T}G] (unknown sample)−C_{T} 18S (unknown sample)]−[C_{T} GI (control sample)−C_{T} 18S (control sample)]; where GI is the gene of interest, e.g. Insl6. Briefly, 2 ng aliquots of total RNA were analyzed using the One-Tube RT-PCR protocol (PE Biosystems). Following reverse transcription at 48° C. for 30 min, the samples were then subjected to PCR analysis using the following cycling parameters: 95° C.×10 rain; 95° C.×15 sec−60° C.×1 min for 40 cycles. Each sample was analyzed in triplicate in individual assays performed on two or more occasions.

Molecular Mechanisms Controlling Expression of Insl6

Transcription of the INSL6 gene is regulated by specific cis-elements and cognate transacting factors. Our data indicate that the INSL6 gene is expressed in a tissue-specific manner. In the mouse expression of this gene is greatest in the testis (pachytene spermatocytes and round spermatids) with lower levels of mRNA detected in kidney, small bowel, heart, brain, and thymus. Knowledge of the factors that regulate expression of Insl6 facilitated the understanding of the biology of this newly identified protein and allowed for the design of strategies to alter its expression in target organs/tissues of the present invention.

As the first step in investigating the molecular mechanisms regulating expression of Insl6 gene, genomic organization of the murine Insl6 gene was mapped. For this purpose genomic clones were isolated by screening a bacteriophage murine genomic library using standard techniques established in the laboratory. Following delineation of exon-intron boundaries by DNA sequencing, the transcription start site(s) were mapped using primer extension and ribonuclease protection assays as previously carried out for the murine growth hormone receptor gene. Determination of the DNA sequence in the vicinity of approximately 500 bases of the transcription start site allowed identification of known promoter motifs e.g., TATA or CCAAT boxes.

The functional role of the 5′-flanking region in the regulation of transcription of the Insl6 gene was assessed by its ability to direct expression of the luciferase gene in transient transfection assays. Briefly, fragments of the 5′-flanking region upstream of the transcription start site(s) were inserted into the promoterless luciferase expression vector pGL3-Basic. The ability of these fusion constructs to exhibit luciferase activity when transiently transfected into various cells such as primary rat spermatocytes, CHO (chinese hamster ovary) and BNL CL.2 (mouse liver) cells was tested. We initiated the analysis by testing the largest suitable fragment in a reporter assay. Transient transfection assays were performed using, such as for example, the transfection protocol LipofectAMINE and calcium phosphate. Primary rat spermatocytes were transfected via a previously established protocol using the GeneFECTOR lipid transfection system (Venn-Nova, Pompano Beach, Fla.) Experimental plasmids were co-transfected with an internal control plasmid constitutively expressing Renilla luciferase and transfection efficiency monitored by the Dual-Luciferase Reporter Assay System (Promega). Expression from experimental plasmids was compared to that from a plasmid containing no promoter sequence and also to a plasmid containing a canonical regulatory element, e.g. CMV and/or SV-40 promoter. In this way, a rough comparison can be made between activities seen in various cell lines, thus allowing for assessment of tissue-selective expression. Our preliminary results indicate that this transcript is most abundant in germ cells with expression being significantly less in kidney, liver, and heart. To identify the regulatory elements controlling tissue-specific expression, we will initially survey for promoter activity in cells representative of these tissues, e.g. primary rat spermatocytes, BNL CL.2 (murine liver), CHO (fibroblast), and CV-1 (monkey kidney). To ascertain that the expression of the reporter gene results from accurate transcription initiation, either ribonuclease protection assay or primer extension assay of the transcribed RNA were carried out as known by those skilled in the art.

Upon demonstration of promoter activity of the 5′-flanking sequence, localization of proximal regulatory region(s) was carried out by deletional analysis of the primary Insl6-luciferase construct. Unidirectional deletions of predictable sizes were engineered by either PCR based methods or by the Exo III/Mung Bean nuclease system. Briefly, for the Exo III/Mung Bean nuclease method, the 5′ upstream regions of the Insl6 gene to be analyzed were cloned into the pBluescript II vector (Stratagene). These clones were double digested to completion with an enzyme that results in a unique 5′ overhanging (e.g., Hind III) or blunt end (e.g., Sma I) and an enzyme that leaves a unique 3′ overhanging end of four bases (e.g., Pst I); the 5′ or blunt restriction site is between the 3′ restriction site and the insert. The double digested DNA was treated with Exonuclease III so that a portion of the insert was made single-stranded and the 5′ single strand was removed by digestion with Mung Bean Nuclease. An aliquot of the deleted DNA was sized by agarose gel electrophoresis, and suitable deletions are selected for further processing. After repair of the ends with Klenow fragment of E. Coli DNA polymerase I, the DNA was circularized and ligated with T4 DNA ligase prior to transformation of competent E. Coli cells. By changing the reaction temperature and the time of incubation, the deletion rate of Exonuclease III can be manipulated to vary the number of nucleotides deleted. The ExoIII/mung Bean method has the advantage over the PCR based technique that it does not need a priori knowledge of the sequence of the DNA that is being manipulated.

Analysis of Protein: DNA Interaction(s) of DNA Regulatory. Sequences

In order to identify the cis-acting elements involved in the regulation of expression of the murine Insl6 gene, the protein:DNA interaction profiles of various DNA fragments containing putative cis-acting elements, were studied using nuclear extracts from a variety of tissues and cell lines (e.g. spermatocytes, liver, kidney). The order in which these DNA fragments were tested for cis-acting elements was guided by the results of comparison of the DNA sequence of these fragments with computer data bases (e.g., MatInspector V2.2) for consensus binding sites for DNA-binding proteins. The precise delineation of DNA sequences that bind sequence-specific proteins was carried out by DNase I protection (footprint) assays and gel retardation assays.

(i) DNase I Footprint Assay—DNA footprinting is a technique to map protein:DNA contact points, and is based on the fact that the presence of protein on the DNA shields that region of the DNA from DNase I catalyzed hydrolysis. Nuclear extracts were prepared by use of protocols known by those skilled in the art. The DNA fragment of interest was end-labeled with ³²P, incubated with nuclear extract, and exposed to low concentration of the endonuclease DNase I so as to introduce random cleavage approximately once for each double-stranded DNA molecule. Cleavage sites are mapped by fractionating the reaction products on a denaturing PAGE sequencing gel, followed by autoradiography. A ladder of bands will result from cleavage at virtually every nucleotide, with a gap (“footprint”) in the pattern corresponding to the region protected by the DNA-binding protein. The nucleotide sequence of this region of the DNA was determined by comparison to a concurrently electrophoresed A+G chemical cleavage reaction. Pilot experiments helped to optimize conditions for random cleavage of the labeled DNA. To determine protein:DNA contact points on each strand of DNA, separate footprint assays were performed with each strand of DNA uniquely labeled. DNase I footprinting was successfully used to map cis-acting elements of the mouse growth hormone receptor gene.

(ii) Gel Retardation Assay—The gel retardation assay resolves DNA/protein complexes from unbound DNA on the basis of differences in electrophoretic mobility through a low percentage, non-denaturing polyacrylamide gel. The DNA/protein complex will, by virtue of its increased size, migrate slower and thus have an apparent molecular weight greater than unbound DNA. The DNA fragment to be tested is labeled with ³²P, incubated with a nuclear protein extract, fractionated by electrophoresis and subjected to autoradiography. Non-specific DNA-protein interactions are minimized by the addition of bovine serum albumin and DNA polymers (e.g., poly [dI.dC]) in the binding reaction, while the specificity of the interaction is confirmed by the addition of unlabeled sequence-specific DNA which compete for DNA-binding protein(s) in a dose-dependent manner. Increasing amounts of unlabeled DNA with the same sequence as the labeled DNA fragment should result in a decrease in the radioactive signal obtained from the DNA/protein complex. The gel retardation assay is a rapid and sensitive method for detecting and analyzing sequence specific DNA-binding protein with its major limitation being the size of the probe (maximum usually in the range of 100-200 bp) that can be employed in this assay. Consequently, the DNA footprinting studies, which can map larger stretches of DNA, preceded this series of experiments and helped identify the specific regions of DNA to be subjected to gel retardation studies as known by those skilled in the art.

Germ cells were fractionated from adult rat testis by unit gravity sedimentation through a 2-4% bovine serum albumin (BSA) gradient using a STAPUT sedimentation chamber (Johns Scientific). Due to differences in sedimentation velocity, germ cells were collected in a predictable sequence. Collected cell fractions were analyzed by differential interference contrast light microscopy and fractions (80-90% pure) containing similar germ cell types were pooled. Using this protocol differences in CREB and CREM protein levels in various germ cell fractions have been shown.

Transfection of spermatocytes has proven to be technically challenging and it is only recently that this has been achieved with consistency and reasonable efficiency. Northern analysis and TaqMan assays (FIGS. 6A & 7) suggested that the Insl6 gene was expressed in a tissue-specific manner. FIG. 7 shows the Insl6 levels measured by TaqMan assay for various murine tissues as described herein. In order to investigate the molecular basis for this tissue-specificity, the reporter constructs in a variety of cell lines derived from different tissues were evaluated [e.g. primary rat spermatocytes, BNL CL.2 (murine liver), CHO (fibroblast), CV-1 (monkey kidney), and primary (fetal and adult) rat hepatocytes]. Leads obtained by this strategy were followed by studies, such as DNase I footprinting, gel shift assay, aimed at localizing the particular cis-element involved in the tissue-specific expression. Clues to the identity of cognate trans-acting factors were confirmed by assays, such as super-shift gel shift assays. The functional significance of the identified trans-acting factors with respect to Insl6 expression were assessed by over-expression studies in transient transfection experiments. It should be noted that in certain instances high levels of the endogenous protein may not allow for the testing of the effect of overexpression of the protein on the activity of the Insl6 promoter. In these instances alternate strategies such as the decoy nucleotide strategy may have to be employed to test the functional significance of the particular trans-acting factor. In this approach high concentrations of double stranded oligonucleotide encoding the recognition motif for the transcription factor of interest are cotransfected into the cell along with the reporter constructs. This decoy oligonucleotide competes with the DNA-binding site in the promoter element and thus decreases the protein available to bind to the promoter element. This strategy was used to study the functional role of the Sp1 and Sp3 family of proteins on the murine growth hormone receptor promoter.

In summary, the expression of Insl6 is increased by cAMP (FIG. 9A). The canonical pathway in cAMP-mediated regulation of gene expression is the activation of cAMP response element binding protein (CREB) and the cAMP responsive element modulator protein (CREM). It is known that CREB is not expressed in germ cell of the testis. Thus it is likely that cAMP's role in regulating Insl6 gene expression is mediated by cAMP-regulated transcription factors other than CREB and CREM such as AP-2, ATF-1, and NF-kB. Initially, clues to the identity of the factors involved in cAMP regulation of Insl6 gene expression were sought by analysis of the DNA sequence of the promoter region of the Insl6 gene. The leads obtained were pursued for identification of the individual factor(s) by techniques such as EMSA, super-shift assays, and transfection experiments. As will be understood by those skilled in the art, the precise choice and sequence of experimental techniques employed necessarily depend on the individual candidate transcription factor being investigated.

Mutation in Codon 8 of INSL6 Gene Results in Conservative Change from Serine to Threonine

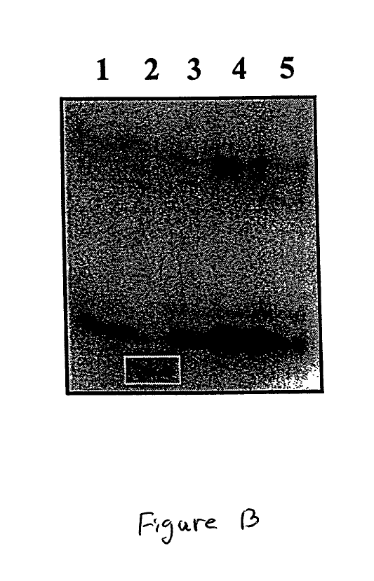

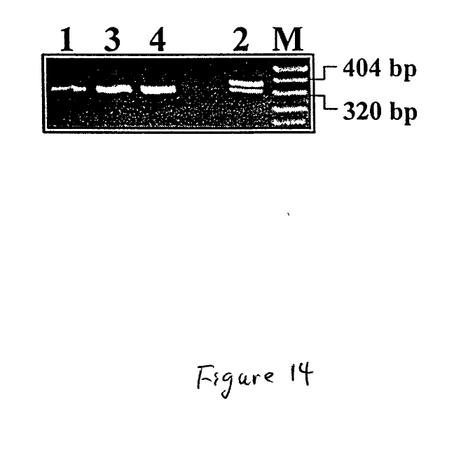

The INSL6 gene is expressed in spermatocytes, intestine, kidney, and vomero-nasal-epithelium. All of these cells are ciliated cells, thus the effect of the INSL6 gene on sperm motility. Mutations in patients with infertility characterized by decreased sperm motility have been characterized. Single strand conformational poymorphism (SSCP) analysis was used to determine DNA sequence variants of INSL6 gene in genomic DNA samples obtained from patients with male infertility associated with decreased sperm motility. Using SSCP techniques a unique conformer in exon 1 of the INSL6 gene. Direct sequencing of the conformer showed a change from T to A at nucleotide position 22. Restriction digest analysis confirmed the presence of this variant as shown in FIG. 13. The variant is predicted to alter serine to threonine at codon 8. Data obtained from SCCP, DNA sequence, and restriction fragment length polymorphism analyses shown in FIGS. 13 and 14 indicate that the subject (patient #2) is a heterozygous carrier of this variant. Analysis of genomic DNA samples from 100 healthy controls (200 alleles) did not reveal the presence of a similar nucleotide change, suggesting that this nucleotide change represents a deleterious mutation despite being a conservative amino acid substitution.

Gene Therapy Application to Reverse INSL6 Gene Mutations

Successful gene therapy generally requires the integration of a gene able to correct the genetic disorder, in this instance a mutation in the INSL6 gene as described above, into the host genome, where it would co-exist and replicate with the host DNA and be expressed at a level to compensate for the defective gene. Ideally, the disease would be cured by one or a few treatments, with no serious side effects. There have been several approaches to gene therapy proposed to date, each of which may benefit from combination with the INSL6 gene useful in the method of the present invention.

One approach is to transfect DNA containing the INSL6 gene of interest into cells, e.g. by permeabilizing the cell membrane either chemically or physically. This approach is generally limited to cells that can be temporarily removed from the body and can tolerate the cytotoxicity of the treatment (i.e. spermatozoa). Calcium phosphate precipitation, DEAE-dextran, electroporation, and direct microinjection are examples of such methods used in mammalian cell transfection.

Liposomes or protein conjugates formed with certain lipids and amphophilic peptides can be used for in vivo and in vitro transfection and DNA coupled to polylysine-glycoprotein carrier complex may also be used. The efficiency of gene integration in this manner is generally very low. It is estimated that the gene of interest integrates into the genome of only one cell in 1,000 to 100,000. In the absence of integration, expression of the transfected gene is limited to several days in proliferating cells or several weeks in non-proliferating cells due to the degradation of the non-integrated DNAs.

Known particle bombardment-mediated gene transfer protocols for transfering and expressing genes in brain tissues can be employed as an effective method for gene transfer into such tissues.

Plasmids may be used to directly transfer INSL6 genetic material into a cell, such as a Sertoli cell, or spermatid cell. DNA segments themselves can therefore be used as delivery agents. The technology for using DNA segments has recently been developed and is generally termed “DNA Vaccination” as understood by those skilled in the art. It is known that cells can take up naked DNA and express encoded proteins. Parenteral, mucosal and gene-gun inoculations known by those skilled in the art may also be used.

Another approach that may be used capitalizes on the natural ability of viruses to enter cells, bringing their own genetic material with them. Retroviruses have promise as gene delivery vectors due to their ability to integrate their genes into the host genome, transferring a large amount of foreign genetic material, infecting a broad spectrum of species and cell types and of being packaged in special cell lines. A variety of retroviral vectors may be used, e.g. herpes simplex virus (U.S. Pat. No. 5,288,641, incorporated herein by reference), cytomegalovirus, and the like. A herpes simplex-thymidine kinase (HS-tK) gene has been delivered to brain tumors using a retroviral vector system, where it successfully induced susceptibility to the antiviral agent ganciclovir.

Gene delivery using second generation retroviral vectors has also been reported. Kasahara et al. (1994) prepared an engineered variant of the Moloney murine leukemia virus, that normally infects only mouse cells, and modified an envelope protein so that the virus specifically bound to, and infected, human cells bearing the erythorpoietin (EPO) receptor. This was achieved by inserting a portion of the EPO sequence into an envelope protein to create a chimeric protein with a new binding specificity. Delivery systems such as described above may be used in connection with the present invention. Further methods use other viruses, such as vaccinia virus, defective hepatitis B viruses, adenovirus and adeno-associated virus (AAV), which are engineered to serve as vectors for gene transfer. Although some viruses that can accept foreign genetic material are limited in the number of nucleotides they can accommodate and in the range of cells they infect, these viruses have been demonstrated to successfully effect gene expression. Adenoviruses do not integrate their genetic material into the host genome and therefore do not require host replication for gene expression, making them suitable for rapid, efficient, heterologous gene expression. For example, the use of adenoviruses and AAV, as are described in U.S. Pat. No. 5,139,941, in the present invention are described herein. The present invention may be used in conjunction with these viruses to deliver the active INSL6 gene during treatment.

Site Directed Mutagenesis

Site directed mutagenesis is a technique useful in the preparation of individual peptides, or biologically functional equivalent proteins or peptides, through specific mutagenesis of the underlying DNA. The technique further provides a ready ability to prepare sequence variants by introducing one or more nucleotide sequence changes into the DNA. For instance, the point mutation described above resulting in the conservative change from a Serine to Threonine amino acid change arising from the mutation at nucleotide position number 22 in the nucleic acid, can be corrected with site-directed mutagenesis.

Site-specific mutagenesis allows the production of mutants through the use of specific oligonucleotide sequences which encode the DNA sequence of the desired mutation to be corrected, as well as a sufficient number of adjacent nucleotides, to provide a primer sequence of sufficient size and sequence complexity to form a stable duplex on both sides of the mutation to be corrected. Typically, a primer of about 17 to 25 nucleotides in length is preferred with about 5 to 10 residues on both sides of the junction of the sequence being altered. The technique of site-specific mutagenesis is generally well known in the art. As will be appreciated, the technique typically employs a phage vector which exists in both a single stranded and double stranded form. Typical vectors useful in site-directed mutagenesis include vectors such as the M13 phage. These phages are readily commercially available and their use is generally well known to those skilled in the art. Double stranded plasmids are also routinely employed in site directed mutagenesis which eliminates the step of transferring the gene f interest from a plasmid to a phage.

In general, site directed mutagenesis in accordance with the present invention is performed by first obtaining a single stranded vector or melting apart the two strands of a double stranded vector which includes within its sequence a DNA sequence which encodes INSL6. An oligonucleotide primer bearing the desired mutated sequence is prepared synthetically employing techniques well known by those skilled in the art. This, primer is then annealed with the single-stranded vector, and subjected to DNA polymerizing enzymes such as E. coli polymerase I Klenow fragment, in order to complete the synthesis of the mutation-bearing strand. Thus a heteroduplex is formed wherein one strand encodes the original non-mutated sequence and the second strand encodes the original non-mutated sequence and the second strand bears the desired mutation. This heteroduplex vector is then used to transform appropriate cells, such as germ cells, and clones are selected which include recombinant vectors bearing the mutated sequence arrangement. Suitable techniques are also described, for example, in U.S. Pat. No. 4,888,286.

Promoters as control sequences can also be used with the present invention to increase expression go the INSL6 gene to increase sperm motility either in vitro or in vivo. Expression vectors comprise protein-encoding nucleic acid segments under control of one or more promoters. To bring a coding sequence “under the control of” a promoter, the 5′ end of the transcription initiation site of the transcriptional reading frame is positioned generally between about 1 and about 50 nucleotides “downstream” of (i.e. 3′ of) the chosen promoter. The “upstream” promoter stimulates transcription of the DNA and promotes expression of the encoded recombinant protein. The promoter used to express the INSL6 gene in non motile spermatids is not critical to the present invention. In an example given, the human cytomegalovirus (CMV) immediate early gene promoter has been used which results in the constitutive, high level expression of the foreign gene. However, the use of other viral or mammalian cellular promoters which are well known in the art is also suitable to achieve expression of INSL6.

A number of viral based expression sytems may be used, for example, commonly used promoters are derived from polyoma, Adenovirus 2, and Simian Virus 40 (SV40). The early and late promoters of SV40 virus are particularly useful because both are obtained easily from the virus as a fragment which also contains the SV40 viral origin of replication. Smaller or larger SV40 fragments may also be used. By employing a promoter with well-known properties, the level and pattern of expression of humanized GFP can be optimized. For example, selection of a promoter which is active specifically in certain cell types will permit tissue-specific expression. Such promoters include those such as the liver fatty acid binding (FAB) protein gene promoter specific for colon epithelial cells; the insulin gene promoter, specific for pancreatic cells; the transphyretin, α1-antitrypsin, plasminogen activator inhibitor type 1 (PAI-1) apolipoprotein AI and LDL receptor gene promoters, each directing specific or preferential expression in liver cells. Promoters active in brain tissues include the myelin basic protein (MBP) gene promoter, specific for oligodendrocytes; the glial fibrillary acidic protein (GFAP) gene promoter, specific for glial cells; and the neural-specific encolase (NSE) promoter that is specific for nerve cells.

Furthermore, selection of a promoter that results in response to specific chemical or physiological signals can permit inducible expression of the INSL6 gene to mediate spermatid motility, and so induce fertility in subjects with infertility due to impaired sperm motility or capacitance. Examples of suitable inducible promoters include the PAI-1 cytochrome P450 gene promoters, heat shock protein genes and hormone inducible gene promoters, and the fos and jun promoters inducible by ionizing radiation. As mentioned above, inducible promoters are useful in vivo, e.g. in gene therapy, and in vitro to correct impaired sperm motility. In screening for the presence of a particular compound within a composition, useful groups inducible promoters are those activated by heavy metals; cytochrome P450 gene promoters, activated by a range of toxic compounds; heat shock protein gene promoters such as the hsp70 promoter, which are stimulated by various stresses to name a few examples.

Adeno-associated virus (AAV) is a vector system used in gene therapy because it is non-pathogenic for humans, it has a high frequency of integration, and it can infect nondividing cells, thus making it useful for delivery of genes into mammlian cells both in tissue culture and in whole animals. AAV vectors may be used for delivery. Recombinant AAV vectors have been used successfully for in vitro and in vivo transduction of marker genes and genes involved in human diseases. Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells. In the absence of coinfection with helper virus, the wild type AAV genome integrates through its ends into human chromosome 19 where it resides in a latent state as a provirus. Recombinant AAV (rAAV), however, is not restricted to chromosome 19 for integration unless the AAV Rep protein is also expressed. When a cell carrying an AAV provirus is superinfected with a helper virus, the AAV genome is “rescued: from the chromosome of from a recombinant plasmid, and a normal productive infection is established. AAV has a broad host range for infectivity.

Typically, recombinant AAV virus (rAAV) is made by co-transfecting a plasmid containing the gene of interest flanked by two AAV terminal repeats and an expression plasmid containing the wild type AAV coding seqeunces without the terminal repeats, for example pIM45). The cells are also infected or transfected with adenovirus or plasmids carrying the adenovirus genes required for AAV helper function. RAAV virus stocks made in such fashion are contaminated with adenovirus which must be physically separated from rAAV particles (for example, by cesium chloride density centrifugation). Alternatively, adenovirus vectors containing the AAV coding regions or cell lines containing the AAV coding regions and some or all of the adenovirus helper genes can be used. Cell lines carrying the rAAV DNA as an integrated provirus helper genes can also be used. rAAV are described in U.S. Pat. Nos. 5,139,941 and 4,797,368 incorporated herein by reference.

Adenovirus vectors, and preferably replication defective vectors, may be used in the context of the present invention. For example, as achieved through the deletion of the viral early region 1 (E1A) region such that the virus is competent to replicate only in cells, such as human 293 cells, which express adenovirus early region 1 genes from their cellular genome. This is important because the virus will therefore no kill normal cells that do not express early gene products. Techniques for preparing replication defective adenoviruses are well known in the art and also describe uses of adenovirus. Other than the requirement that the adenovirus vector be replication defective, the nature of the adenovirus vector is not believed to be crucial. The adenovirus may be of any of the 42 different known serotypes or subgroups A-F. Adenovirus type 5 of subgroup C is the preferred starting material in order to obtain the conditional replication-defective adenovirus vector for use in the method of the present invention. This is because Adenovirus type 5 is a human adenovirus about which a great deal of biochemical and genetic information is known, it has historically been used for most contructions employing adenovirus as a vector, and it is non-oncogenic.

In that the vectors for use in these aspects are replication defective, they will typically not have an adenovirus E1 region. Thus it will be most convenient to introduce the INSL6 gene at the position from which the E1 coding sequences have been removed. However, the position of insertion of the INSL6 gene is not critical. The transcriptional INSL6 unit may also be inserted in lieu of the deleted E3 region in E3 replacement vectors.

SEQ ID Nos. 1 and 2 represent the mouse Insl6 DNA and protein sequences, respectively.

SEQ ID Nos. 3 and 4 represent the human INSL6 DNA and protein sequences, respectively.

SEQ ID No. 5 represents the human INSL6 DNA sequence having the point mutation at position 22 changing codon 8 from serine to threonine.

All of the compositions and methods described and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically it will be understood by those skilled in the art that certain agents that are both chemically and physiologically related may be substituted for the agents described herein to achieve the same or similar results. All such similar agents apparent to those skilled in the art are deemed to be within the scope, spirit and concept of the invention as defined by the appended claims.

What is claimed is:

1. A method for modulating male fertility in a male subject comprising regulating the in vivo expression of an INSL6 gene of SEQ ID No. 3.

2. The method of claim 1 comprising administering to said male subject a composition comprising nucleic acids complementary to a coding strand of a cDNA encoding the INSL6 protein of SEQ ID No. 4 for regulating the expression of the INSL6 gene of SEQ ID No. 3 to regulate in vivo expression of the INSL6 gene of SEQ ID No. 3.

3. The method of claim 2 wherein the complementary nucleic acids comprise nucleic acid analogs effecting nascent transcript termination.

4. The method of claim 2 wherein the in vivo expression of INSL6 gene is regulated by administering an effective amount of a repressor of the INSL6 gene.

5. The method of claim 2 wherein the in vivo expression of INSL6 gene is regulated by administering an effective amount of a promoter of the INSL6 gene.

6. A method for enhancing fertility of a male subject comprising the steps of administering an effective amount of Insl6 protein of SEQ ID No. 4 to the subject's sperm during in vitro fertilization to improve capacitance of the sperm.

7. A method for restoring sperm motility of a male subject comprising administering an effective amount of Insl6 protein of SEQ ID No. 4 to the subject's sperm during in vitro fertilization.

8. A method for promoting development in a male embryo comprising delivering to said male embryo an effective amount of a vector comprising a nucleic acid encoding an Insl6 protein or a protein having substantial homology to the protein of SEQ ID No. 4 to induce expression of the INSL6 protein of SEQ ID No. 4.

9. The method of claim 1 wherein the vector comprising the nucleic acid encoding the INSL6 protein of SEQ ID No. 4 is delivered to the testis of said male embryo.

10. The method of claim 1 wherein the vector comprising the nucleic acid encoding the INSL6 protein of SEQ ID No. 4 is delivered to the germ cells of said embryo.

11. A method for regulating levels of INSL6 protein of SEQ ID No. 4 comprising the step of regulating the degradation of INSL6 protein.

12. A method for regulating levels of INSL6 protein of SEQ ID No. 4 comprising the step of enhancing synthesis of INSL6 protein.

13. A composition for modulating male fertility comprising an effective amount of a protein of SEQ ID. No. 2 encoded by an INSL6 nucleic acid of SEQ ID No. 3, and a pharmaceutical carrier wherein said protein is incorporated into said pharmaceutical carrier.

14. A composition for modulating male fertility comprising an effective amount of a substance selected from the group consisting of a repressor of the INSL6 gene, a promoter of the INSL6 gene, a nucleic acid complementary to the cDNA of INSL6 gene, and a nascent-transcript-terminating nucleic acid complementary to the INSL6 gene, and a pharmaceutically suitable carrier.

15. The composition of claim 14 wherein the nucleic acid complementary to the cDNA of INSL6 gene is an anti-sense nucleic acid.

16. An isolated and purified expression vector comprising a control nucleic acid sequence operatively linked to an isolated and purified nucleic acid insert encoding a protein substantially identical to the INSL6 protein of SEQ ID No. 4.

17. The expression vector of claim 16 wherein the control sequence is a promoter enhancing expression of the insert.

18. The expression vector of claim 17 wherein the control sequence is a repressor suppressing expression of the insert.

19. The expression vector of claim 17 wherein the control sequence is modulated by a control molecule selected from a promoter and a repressor.

20. A stably transformed cell line stably expressing a substantially identical protein to a INSL6 protein of SEQ ID No. 4.

21. The cell line of claim 20 wherein the transformation is by transfection with a vector comprising an isolated and purified nucleic acid encoding a protein substantially identical to an INSL6 protein of SEQ ID. No. 4.

22. The cell line of claim 20 wherein the cell line is a mammalian cell line.

23. The cell line of claim 22 wherein the cell line is a mouse cell line.

24. The cell line of claim 22 wherein the cell line is a human cell line.

25. The cell line of claim 22 wherein the cell line is an insect cell line.

26. A method for modulating male fertility comprising mutating the INSL6 gene of SEQ ID No. 3 at nucleotide number 22 whereby codon number 8 is changed from a serine-encoding codon to a threonine-encoding codon.

27. The method of claim 26 wherein the mutating step comprises site-directed mutagenesis of the INSL6 gene of SEQ ID No. 3.

28. The method of claim 26 wherein the mutating step comprises anti-sense control of expression of the INSL6 gene of SEQ ID No. 3.

29. The method of claim 26 wherein the mutating step is performed in vivo. 52.

30. The method for modulating male infertility comprising mutating the INSL6 gene of SEQ ID No. 3 at nucleotide number 22 whereby codon number 8 is changed from a theonine-encoding codon to a serine-encoding codon.

31. The method of claim 30 wherein the mutating step comprises site-directed mutagenesis of the INSL6 gene of SEQ ID No. 3.

32. The method of claim 30 wherein the mutating step comprises anti-sense control of expression of the INSL6 gene of SEQ ID No. 3.

33. An isolated and purified antibody or fragment thereof which specifically binds to a polypeptide of SEQ ID No. 4.

34. The antibody or fragment thereof of claim 33 wherein said antibody is a polyclonal antibody.

35. The antibody or fragment thereof of claim 33 wherein said antibody is a monoclonal antibody.

36. A hybridoma cell line which produces the monoclonal antibody or fragment thereof of claim 35.

37. An isolated and purified antibody or fragment thereof which specifically binds to a polypeptide of SEQ ID No.2.

38. A kit comprising (a) the antibody or fragment thereof of claim 33 and (b) a detection reagent to detect the presence of a specifically bound antibody.

39. A composition comprising the antibody or fragment thereof of claim 33 and a pharmaceutically acceptable carrier, excipient or diluent.

40. The kit of claim 38 wherein said detection reagent is a radioisotope, affinity label, enzymatic label, fluorescent label, or paramagnetic label.

41. A method of detecting the presence of an INSL6 polypeptide having the amino acid sequence of SEQ ID No. 4 or a fragment thereof in a cell, tissue, or fluid sample, comprising:
(a) contacting said cell, tissue, or fluid sample with an antibody or fragment of claim 33 under conditions that allow the formation of an antibody/polypeptide complex, and
(b) detecting the presence of an antibody polypeptide complex, wherein the presence of the antibody/polypeptide complex indicates the presence of an INSL6 polypeptide.

42. A method of modulating male fertility comprising modulating cyclic-AMP levels to modulate INSL6 activity in sperm cells.

43. A method for modulating male fertility comprising modulating a proteasome pathway to modulate INSL6 activity.

44. The method of claim 43 wherein the proteasome pathway is modulated in vivo.

45. The method of claim 43 wherein the proteasome pathway is modulated in vitro.

46. The method of claim 42 wherein the cyclic-AMP level is modulated in vivo.

47. The method of claim 42 wherein the cyclic-AMP is modulated in sperm cells in vitro.

48. A method of diagnosing a mutation or deletion in a the INSL6 sequence of a subject comprising determining allelic homozygocity or heterozygocity of said subjects INSL6 by hybridizing in a Southern blot with marker probe selected from the group consisting of D9S251, D9S248, D9S256, DS1686, D9S1873, D9S1792, and D9S1858.

49. A method for modulating male fertility in a male subject comprising regulating the in vivo expression of an insulin 6 gene.

50. The method of claim 49 wherein the in vivo expression of the insulin 6 gene is regulated by administering an effective amount of a promoter of the insulin 6 gene.
